(19) United States
(12) Patent Application Publication
(10) Pub. No.: US 2023/0075772 A1
(43) Pub. Date: Mar. 9, 2023

(54) IMPLANTABLE BONE SCAFFOLDS INCLUDING AT LEAST ONE INTEGRATION AID, METHODS OF MAKING AND USING THE SAME

(71) Applicant: UNIVERSITY OF UTAH RESEARCH FOUNDATION, Salt Lake City, UT (US)

(72) Inventors: Sujee Jeyapalina, Salt Lake City, UT (US); Jayant P. Agarwal, Salt Lake City, UT (US); Jill E. Shea, Salt Lake City, UT (US); James Peter Beck, Salt Lake City, UT (US); Clark Richard Nielson, Salt Lake City, UT (US); Samantha Steyl, Charlotte, NC (US)

(21) Appl. No.: 17/900,240

(22) Filed: Aug. 31, 2022

Related U.S. Application Data
(60) Provisional application No. 63/239,749, filed on Sep. 1, 2021.
(60) Provisional application No. 63/300,742, filed on Jan. 19, 2022.

(51) Int. Cl. C12N 5/0775; A61K 35/12

(52) U.S. Cl. CPC C12N 5/0662; A61K 35/12; C12N 2506/1384

(57) ABSTRACT

Embodiments disclosed herein relate scaffolds containing fluoridated apatites sintered at a temperature of at least 950° C. and with at least one integration aid to increase integration of the scaffold in a patient, as well as methods of making and using the same.

### CROSS-REFERENCE TO RELATED APPLICATIONS

This application claims priority to U.S. Provisional Patent Application No. 63/239,749 filed on Sep. 1, 2021 and U.S. Provisional Patent Application No. 63/300,742 filed on Jan. 19, 2022, the disclosure of each of which is incorporated herein, in its entirety, by this reference

### BACKGROUND

Implantable engineered bone scaffolds with auto-graft-like properties (referred to as implantable scaffolds) may be surgically implanted into the tissue(s) of a subject, such as a human or animal. Implantable scaffolds often fail because the implant fails to integrate with the surrounding bone tissues.

The long-term success of dental implants is dependent upon sustained osseointegration. Without sufficient bone to support the implant placement, loosening will occur, increasing the risk of biomechanical overload and/or implant fracture, which often require implant removal and re-implantation.

### SUMMARY

Embodiments disclosed relate to implantable scaffolds including at least one integration aid and methods of making and using the same. In an embodiment, an implantable scaffold is disclosed. The implantable scaffold includes a fluoridated apatite structure sized and shaped for implantation in an animal. The fluoridated apatite structure defines a plurality of pores. The implantable scaffold also includes at least one integration aid including at least one of stromal vascular fraction adhered to the fluoridated apatite structure or at least one metal substitute substituted into the fluoridated apatite structure, the at least one metal substitute including one or more of zinc, silver, or iron.

In an embodiment, a method of making an implantable scaffold is disclosed. The method includes providing fluoridated apatite particles, sintering the fluoridated apatite particles at a sintering temperature of at least 950° C. to form a fluoridated apatite structure, and introducing at least one integration aid into the fluoridated apatite structure. The at least one integration aid includes at least one of stromal vascular fraction adhered to at least a portion of the fluoridated apatite structure or at least one metal substitute substituted into the fluoridated apatite structure, the at least one metal substitute including one or more of zinc, iron, or silver.

In an embodiment, a method of using an implantable scaffold. The method includes providing an implantable scaffold. The implantable scaffold includes a fluoridated apatite structure sized and shaped for implantation in an animal. The fluoridated apatite structure defining a plurality of pores. The implantable scaffold also includes at least one integration aid including at least one of stromal vascular fraction adhered to at least a portion of the fluoridated apatite structure or at least one metal substitute substituted into the fluoridated apatite structure, the at least one metal substitute including one or more of zinc, iron, or silver. The method also includes implanting the implantable scaffold in a subject.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a side cross-sectional view of a scaffold disposed in a bone, according to an embodiment.

FIG. 2 is a flow diagram of a method of making a scaffold, according to an embodiment.

FIG. 3 is a flow chart of a method of using a scaffold having fluoridated apatite, according to an embodiment.

FIG. 4 is a micro-CT image of the porous structure of an FA material (top) and a HA material (bottom).

FIG. 5 is a scanning electron microscopy image of an FA powder as synthesized.

FIG. 6 illustrates X-ray diffraction patterns of (1) as-made FA powder, (b) FA sintered at 1250° C., (c) FA sintered at 1350° C., (d) FA sintered at 1450° C., and (e) an HA reference pattern.

FIG. 7 is a set of SEM images of the implantable scaffolds before sintering and after sintering at various temperatures, showing the formation of micro-structured surface topography between 1150 and 1200° C.

FIG. 8 is a representative histogram (left) of the pore size distributions for five foam-casted porous scaffolds and representative images of micro-CT scans (right) used to generate the histogram.

FIG. 9 is a graph illustrating the calculated compression strengths for several foam-casted scaffolds sintered at various temperatures.

FIG. 10 is a graph illustrating the days 2 and 10 RT-PCR, mRNA expression data for osteogenic markers, Runx2 (left) and SPP1 (right).

FIG. 11 includes images showing the osteoblast markers expressed at 2 and 10 days post-seeding.

FIG. 12 is a representative set of fluorescence-activated cell sorting data of the freshly harvested human stromal vascular fraction cells.

FIG. 13 is a graph illustrating the principal component (PC) analysis of stromal vascular fraction cells grown on a control surface (cell culture flask) and FA surface, post 11 days of culturing.

FIG. 14 is a volcano plot showing the differentially expressed genes between FA and control.

FIG. 15 is a set of box plots showing the most differentially expressed genes expressed by stromal vascular fraction seeded on the FA and control surfaces, post 11 days of culturing.

FIG. 16 is representative images showing osteoblast marks, osteocalcin (OCN), and osteopontin (OPN,) expressed after 11 days post-culturing.

FIG. 17 is a representative set of micro-CT images of the femoral defect at necropsy (12 weeks, post-implantation).

FIG. 18 is a box plot showing the percent of new bone within the defect.

FIG. 19 is a representative set of photomicrographs of bone defects with and without scaffolding stained with Sanderson's Rapid Bone Stain™ and magnified images of the top panel are given in the bottom panel.

FIG. 20 is representative photomicrographs showing the multi-nucleated cells (possible osteoclasts, shown with arrows) adjacent to the FA surface (right) and a representative microphotograph of the resorbing (degraded) HA scaffolding (left), 12 weeks post-implantation.

FIG. 21 is representative highly magnified photomicrographs showing the multi-nucleated cells osteoclast (indicated with arrows) adjacent to the FA surface (right) and within a cluster of HA surfaces (left).

FIG. 22 is a representative set of SEM images showing FA and 1% and 2% Zn-doped foam-casted FA surfaces sintered at 1100° C., 1150° C., and 1200° C.

FIG. 23 is a representative photograph of the foam-casted porous FA scaffolding (left) and two 2D slices from a micro-CT scan.

FIG. 24 is representative SEM images showing the surface morphology of several of the disks sintered at 1150° C. having different zinc content and different compressive loads.

FIG. 25 is a bar chart showing the number of adherent bacteria on zinc substituted disks sintered at 1150° C. after 48 hours in a biofilm reactor.

FIG. 26 is representative SEM images showing the adherent biofilm after 48 hours in a biofilm reactor.

FIG. 27 is a representative set of confocal images showing the nucleus and osteocalcin 10-days post-seeding.

FIG. 28 is a bar graph showing the percent of new bone within the scaffolding after 12 weeks in situ in rats.

FIG. 29 is a representative micro-CT image of 2.0 molar % Zn-FA scaffold encapsulated by bone matrix post-10 weeks in situ.

### DETAILED DESCRIPTION

Embodiments disclosed relate to implantable scaffolds including at least one integration aid and methods of making and using the same. An example, implant includes a fluoridated apatite structure sized and shaped for implantation in an animal (e.g., a human). The implantable scaffold also includes at least one integration aid configured to improve osseointegration with the implantable scaffolds. In an embodiment, the integration aid includes stromal vascular fraction adhered to (e.g., disposed in and/or on) the fluoridated apatite structure. It has been found that stromal vascular fraction facilitates bone regrowth and integration of the implantable scaffold with the native bone tissue of the subject. In an embodiment, the integration aid includes metal substitute (e.g., zinc, iron, or silver) substituted into the fluoridated apatite structure. It has been found that the metal substitute substituted into the fluoridated apatite structure provided antibacterial properties to the fluoridated apatite structure thereby minimizing bacteria on the fluoridated apatite structure that may impede new bone deposition and promotes cell differentiation (e.g., direct stem cells to osteogenic lineage) relative to an implantable scaffold that only includes FA.

An ideal engineered bone substitute should replicate the beneficial qualities of autograft bone, including structural support, a reliable source of osteogenic cells, and the capacity to generate osteogenic signals. One biomaterial that has been used clinically as a structural and biological bone graft substitute is synthetic hydroxyapatite [Ca₁₀(PO₄)₆(OH)₂] (“HA”). The HA scaffold has the advantage of possessing a similar chemical structure of the mineral component of native bone tissue stoichiometrically-making the HA biocompatible. HA substitutes, however lack necessary mechanical strengths to be an adequate replacement to autograft. In the hope of improving the mechanical and degradation properties of apatite-based scaffolds, the crystallinity of the HA may be improved by fluoridation and heat treatment. The resultant, fully fluoridated apatite particles may be similar to HA but has enhanced properties in terms of biocompatibility, strength, in vivo stability, and cell adhesion. The fluoridated apatite particles have also been shown to produce comparable mechanical strengths (4.17 to 13.5 MPa), upon sintering at high temperatures, to human cancellous bone tissue.

The fluoridated apatites (e.g., fluoridated apatite particles and fluoridated apatite structures) disclosed herein include one or more of fluorohydroxyapatite (“FHA”) or fluorapatite (“FA”) that is sintered at a sintering temperature selected to provide a desired surface morphology for the scaffold. FHA (Ca₁₀(PO₄)₆F_{y}(OH)_{2-y}) and FA (Ca₁₀(PO₄)₆F₂) are partially and fully fluoridated forms of apatite (e.g., HA). The scaffolds disclosed herein including fluoridated apatite demonstrate excellent adhesion to tissue cells relative to implants or scaffolds formed from other materials such as HA. The scaffolds disclosed herein may be used as bone grafts, such as a bone substitute or carrier for the same. Proper integration of the bone with the implant surface is important for maintaining a stable interface and preserving implant integrity.

The fluoridated apatite structure of the scaffolds herein are made of nonbiological materials (e.g., materials foreign to the body's internal environment) that may be used as bone grafts. Conventional bone grafts may include autografts or allografts. Autografts have no immunogenic response, but it has a limited supply. Decellularized allografts harvested from cadaveric sources have the advantage of being osteoconductive and osteogenic; however, they can be associated with risk of infectious disease immunogenicity, host rejection, and accelerated graft resorption. Allografts have no autologous cells and require cells to migrate in, which takes time, during which time large portion of grafts may resorb and lose strength and structure, which may cause failure of the allografts. Bone substitutes have been developed in response to the shortcomings of autografts and allografts. Bone substitutes have focused on providing the necessary matrix to support bone-ingrowth/ongrowth and integration by providing a biocompatible, bioresorbable, and porous scaffold made from materials such as HA, collagen, and biodegradable synthetic materials.

Conventional scaffolds that have integrated extracellular matrix proteins or growth factors, typically BMPs, have certain limitations, such as the uncontrolled release of growth factors, which leads to the formation of bone in undesirable areas of the body (e.g., ectopic bone formation). In contrast, the autograft-like living bone substitutes of the scaffolds disclosed herein may incorporate cell types that the patient's own body already utilizes in response to bone loss or physical insult. As the scaffold material (e.g., FA and FHA) is naturally resorbed within the body over time, it is replaced with new bone tissues that may be stimulated within the porous structure by the addition of the integration aids disclosed herein. Moreover, the use of fluoridated apatite structures results in a relatively slower resorption rate than in conventional bone grafts, thereby mitigating the undesirable premature release of growth factors described above.

The scaffolds disclosed herein are sized and shaped for implantation in an animal (e.g., human). For example, the scaffolds disclosed herein can be custom fabricated (e.g., 3-D printed) to fit the precise size and shape of a defect a patient presents with. The scaffolds disclosed herein eliminate requirements for autograft donor sites, a second surgery to harvest grafts, pain associated with graft removal, and prolonged recovery time. The scaffolds disclosed herein may also be sterile, off-the-shelf products that may be opened in the operating room by the surgical team such as immediately prior to implantation.

Autograft-like porous bone scaffolds are described herein. They may be fabricated with a mineral matrix (e.g., fluoridated apatite structures) and integration aids (e.g., agents configured to cause or otherwise facilitate de novo osseous tissue repair and regeneration). As the scaffold is naturally resorbed over time, it is replaced with an influx of new bone formation, due in part to the integration aids. Accordingly, the scaffolds disclosed herein are particularly useful in the orthopedic, plastic surgery, and dental fields as customizable scaffold material to repair instances of bone loss, defects, and trauma.

As previously discussed, the implantable scaffolds disclosed herein include at least one integration aid. The integration aids disclosed herein (e.g., stromal vascular fraction and the metal substitute) provide additional properties to the fluoridated apatite structure that facilitate integration of the implantable scaffold. For example, it is currently believed that the implantable scaffolds disclosed herein that include one or both of the integration aids disclosed herein may perform similar to autografts.

In an embodiment, the integration aids include a stromal vascular fraction. The stromal vascular fraction may be deposited in the pores of the fluoridated apatite structure such that at least some of the pores are at least partially occupied by the stromal vascular fraction. The stromal vascular fraction may also be deposited on other surfaces of the fluoridated apatite structure. It has been found that stromal vascular fraction may have the capacity to regenerate osseous tissue to a level comparable with autograft bone. Stromal vascular fraction includes a combination of cell types. In an example, the stromal vascular fraction may include adipose-derived stem cells (“ADSC”) along with other cell types. In an example, the stromal vascular fraction includes progenitor stem cells since such cells can differentiate into multiple lineages that can be differentiated on the osteogenic lineage. In an example, the stromal vascular fraction may include perivascular cells, leukocytes, endothelial cells, fibroblasts, progenitor stem cells, ADSC, other adipose cells, or combinations thereof. In an embodiment, the stromal vascular fraction may include minimally processed stromal vascular fraction (i.e., stromal vascular fraction provided in an extraction process that involves minimal manipulation of the fat source of respective patients)

The stromal vascular fraction may also represent an improvement over implantable scaffolds that include growth factors and other stem cells instead of stromal vascular fraction. For example, growth factors like bone morphogenetic proteins (“BMP”) have been shown to promote bone growth at injury sites and differentiate stem cells into a bone lineage. However, BMP has a disadvantage that their bioavailability may decrease over time and may have a short half-life. In another example, BMP and osteoblasts require the implant scaffold to exhibit rough surfaces to maximize their benefit. ADSC express bone lineage markers on the implant scaffold but the timing of the expression is dependent upon the type of material forming the implantable scaffold. However, stromal vascular fraction may not have at least some of these issues associated with osteoblasts, and ADSC, for example, because the stromal vascular fraction may include a plurality of cell types that will contribute to neo-vascularization. Further, it has been found that stromal vascular fraction significantly encourages stem cell differentiation towards osteogenic lineage on FA surfaces.

The autologous adipose-derived stromal vascular fraction (i.e., the stromal vascular fraction is derived from tissue of the subject that receives the bone graft) can be obtained at the time of the surgery from the patient. Adipose-derived stromal vascular fraction is an ideal stem cell source in a surgical setting as stromal vascular fraction can be extracted from local adipose tissues and administered with bone scaffolds. For example, the stromal vascular fraction may be obtained from the fat tissue (e.g., from liposuction) of the subject that receives the bone scaffold. The adipose-derived stromal vascular fraction may generate osseous tissue better than other stem cell types. In other words, the adipose-derived stromal vascular fraction may cause the implantable scaffold to behave more like an autograft than other stem cell populations. In an embodiment, the stromal vascular fraction may be formed from (i.e., isolated from) the breakdown of adipose tissue either by enzymatic or mechanical techniques. In other words, stromal vascular fraction may be obtained from the subject through minimal manipulation of the adipose tissue and the stromal vascular fraction can be isolated in the same operative setting as the reconstruction of the bone.

In an embodiment, the implantable scaffolds may include about 1×10² or more stromal vascular fraction cells, such as about 1×10³ or more, about 1×10⁴ or more, about 1×10⁵ or more, about 1×10⁶ or more, about 1×10⁷ or more, about 1×10⁸ or more stromal vascular fraction cells/cm². The amount of stromal vascular fraction cells may depend on the size of the implantable scaffold and/or the number of stromal vascular fraction cells removed from the patient.

In an embodiment, the integration aid includes at least one metal substitute substituted (e.g., doped) into the fluoridated apatite structure. The metal substitute may include zinc, iron, silver, any other biocidal metal, or any other suitable metal. It has been found that the presence of the metal substitute in the fluoridated apatite structure improves the antimicrobial properties of the implantable scaffold without increasing the cell toxicity of the implantable scaffold. Further, it has been found that the presence of the metal substitute in the fluoridated apatite structure increases cell differentiation after implantation compared to non-metal substituted fluoridated apatite structures. In a particular example, the metal substitute includes zinc because natural zinc substituted HA/FHA is present in the bone and enamel of human teeth. It has also been found that zinc has a stimulatory effect on cells.

The metal substitute may replace some of the calcium in the fluoridated apatite structure. For example, when the fluoridated apatite structure includes FA, the chemical formula of the FA may be FA Ca_{(10-X)}(PO₄)₆F₂Z_{X}, where Z the metal substitute. When the fluoridated apatite structure includes FHA, the chemical formula may be Ca_{(10-X)}(PO₄)₆F_{y}(OH)_{2-y}Z_{X}, where Z is the metal substitute. X in either chemical formula may be selected such that 0.25 molar % to about 15 molar % of the calcium is replaced with the metal substitute, such as in ranges of about 0.25 molar % to about 0.75 molar %, about 0.5 molar % to about 1 molar %, about 0.75 molar % to about 1.5 molar %, about 1 molar % to about 2 molar %, about 1.5 molar % to about 2.5 molar %, about 2 molar % to about 3 molar %, about 2.5 molar % to about 3.5 molar %, about 3 molar % to about 4 molar %, about 3.5 molar % to about 4.5 molar %, about 4 molar % to about 5 molar %, about 4.5 molar % to about 6 molar %, about 5 molar % to about 7 molar %, about 6 molar % to about 8 molar %, about 7 molar % to about 9 molar %, about 8 molar % to about 10 molar %, about 9 molar % to about 12 molar %, or about 10 molar % to about 15 molar %. The metal substitute may replace the calcium in the FA and FHA during the synthesis of the FA and FHA and/or during the formation of the fluoridated apatite structure. It is noted that the chemical formulas of the FA and FHA including the metal substitute may be slightly different from the chemical formulas provided above because the substitution of the metal substitute into the FA and FHA may slightly change the stoichiometry of the FA and FHA. It is noted that the molar % of calcium that is replaced with the metal substitute may be selected to be less than 5 molar % to prevent secondary phase formations in the hydroxyapatites.

The implantable scaffolds disclosed herein that include the metal substitute represent an improvement over “non-metal substituted implantable scaffolds.” Examples of non-metal substituted implantable scaffolds include decellularized cadaveric bone tissues, synthetic polymeric-based engineered bone grafts. Such non-metal substituted implantable structures exhibit no natural antimicrobial properties. Thus, the non-metal substituted implantable scaffolds might not be able to limit infection of the contaminated implantation site which, in turn, prevents osseous tissue ingrowth and integration and results in failed bone implants. To remedy this situation, the non-metal substituted implantable scaffolds may be cleaned prior to implantation but, due to the porosity thereof, it may be difficult to completely sterilize the implantable scaffold prior to implantation. The non-metal substituted implantable scaffolds may also have an antimicrobial agent disposed in the pores thereof but the antimicrobial agent may limit the volume available for other agents (e.g., stromal vascular fraction) and may inhibit integration of the non-metal substituted implantable scaffolds into the tissue. However, it has been found that the implantable scaffolds that include the metal substitute exhibit antimicrobial properties that at least inhibit contamination of the implantation site. Further, it has been found that the antimicrobial properties of the implantable scaffolds that include metal substitute exhibit enhanced cell differentiation and do not increase the cell toxicity of the implantable scaffold.

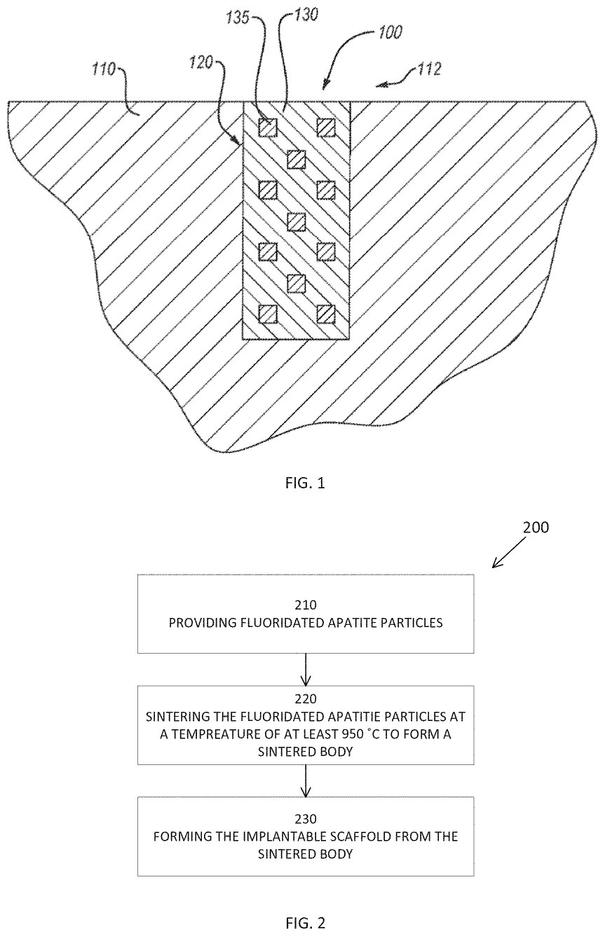

FIG. 1 is a side cross-sectional view of a scaffold 100 disposed in a bone 110, according to an embodiment. The scaffold 100 may be disposed in the bone 110 or other tissue of a patient, such as at a wound site 112 (e.g., implantation site). The tissue may include one or more tissues, such as soft tissue (e.g., skin), hard tissue (e.g., bone), or combinations thereof. As shown, a pocket 120 extends into the bone 110 at the wound site 112. The pocket 120 may be formed by an injury or surgical intervention. For example, an area within a jaw bone may be cleared or shaped to make room for an implant, such as to build up bone for implantation of a dental implant. Similarly, space in a hip, a femur, spine, etc. may be formed to receive a scaffold to replace damaged/diseased bone tissue. The scaffold 100 may be positioned within the wound site 112, such as in one or more of soft tissue or hard tissue (e.g., bone).

The scaffold 100 serves as a structure that provides mechanical strength, a substrate for bone growth. The bulk structure of the scaffold 100 is formed of fluoridated apatite such as one or more of FA or FHA (e.g., FA or FHA including the metal substitute). The fluoridated apatite structure of the scaffold 100 may be a porous sponge-like (though substantially rigid) structure. The fluoridated apatite structure of the scaffold 100 may be framework, block, rod, plug, wedge, or any other structure formed from a plurality of fluoridated apatite particles and has a plurality of pores therein. The bulk structure of the scaffold may be shaped to fit into a selected space or cavity, within a subject's body. At least some of the pores in the bulk structure of the scaffold may be formed by casting fluoridated apatite material in an investment material and removing the investment material, such as by one or more of dissolution, combusting, heating, machining, lasing, or any other suitable technique. At least some of the pores in the scaffold 100 (e.g., in the microstructure) may be due to the crystalline nature of the fluoridated apatite of the scaffold 100. In an embodiment, as shown in FIG. 1, the structure of the scaffold 100 only includes the fluoridated apatite structure. Examples of structures of the scaffold 100 that only include the fluoridated apatite structure are disclosed in U.S. patent application Ser. No. 17/420,589 filed on Jan. 9, 2020, the disclosure of which is incorporated herein, in its entirety, by this reference. In an embodiments, the structure of the scaffold 100 may include a body that is distinct from the fluoridated apatite structure. In such an embodiment, the fluoridated apatite structure may be a coating disposed on the fluoridated apatite structure. Examples of structures that may have a body that is distinct from the fluoridated apatite structure are disclosed in U.S. patent application Ser. No. 17/420,579 filed on Jan. 9, 2020, the disclosure of which is incorporated herein, in its entirety, by this reference.

The scaffold 100 defines a plurality of surfaces that can bond to bone or other tissues and may provide a substrate through which dopants may be delivered to the implantation site. The scaffold 100 may include one or more void spaces 130 therein. The void spaces 130 may be pores. In some embodiments, the void spaces 130 may include pores or chambers formed (e.g., molded, machined, dissolved, etc.) in the bulk structure of the scaffold 100.

The scaffold 100 may include at least one integration aid. For example, one or more surfaces of the scaffold 100 may have the stromal vascular fraction 135 disposed thereon or disposed in the void space (e.g., pores) of the scaffold 100. The one or more void spaces 130 may be at least partially filled with the stromal vascular fraction 135 configured to promote bone growth that are distinct from the stromal vascular fraction. Alternatively or additionally, the void spaces 130 may be at least partially filled with dopants. Suitable dopants may include collagen, keratose, differentiation promoters (e.g., BMP-2), platelet-rich plasma, stem cells (e.g., ADSCs), demineralized bone matrix, and the like.

The scaffold 100 may be formed in any shape (e.g., size and dimensions) for implantation into the tissues (e.g., hard and/or soft tissues) of a subject. For example, the scaffold 100 may be sized and shaped to form a post, a screw, a joint, a socket, a ball, or any other bone structure. In embodiments, the scaffold 100 may be disposed on or sized and shaped to host percutaneous implant such as percutaneous osseointegrated (OI) prosthetics, dental implants, orthopedic implants, or the like.

The fluoridated apatite in the scaffold 100 provides a medium for preferential attachment of tissue cells (e.g., osteoblast cells, epithelial cells, etc.) to the scaffold 100. The scaffold 100 includes fluoridated apatite material such as, FHA, FA, or combinations thereof. For example, the scaffold 100 may consist of or consist essentially of FA, FHA, one or more integration aids, one or more optional dopants 135, or combinations of any of the foregoing. In embodiments, the scaffold 100 may consist of or consist essentially of FA and one or more integration aids. FA has proven to be particularly effective at adhering to the osteoblast cells. FA in the scaffold 100 promotes tissue adhesion which provides a substrate for new bone growth, and which reduces or eliminates gaps along the surfaces of the scaffold, thereby reducing or eliminating infections at the wound site 112 (e.g., implant site). The scaffolds disclosed herein increase tissue bonding and growth at the surface of the scaffold. The scaffolds disclosed herein reduce or eliminate downgrowth of osteoblast cells, epithelial cells, or other local cells along the scaffold surface relative to conventional scaffolds (e.g., scaffolds that do not have the fluoridated apatite structure disclosed herein). Osteoblast cells showed great affinity for fluoridated apatite surfaces that were sintered at 1050° C. to 1250° C. when compared to HA and titanium surfaces.

The material that forms the fluoridated apatite structure may include FA and/or FHA particles that are pressed into a green body. For example, to form the green body, the material that forms the fluoridated apatite structure may be disposed in a mold and be pressed (i.e., have a compressive load applied thereto). The compressive load may be about 25 MPa or greater, about 30 MPa or greater, about 45 MPa or greater, about 60 MPa or greater, about 75 MPa or greater, about 90 MPa or greater, about 105 MPa or greater, about 120 MPa or greater, or in ranges of about 30 MPa to about 60 MPa, about 45 MPa to about 75 MPa, about 60 MPa to about 90 MPa, about 75 MPa to about 105 MPa, or about 90 MPa or greater. It has been unexpectedly found that the compressive load used to form the green body affects the antimicrobial properties of the fluoridated apatite structure, for example, when the fluoridated apatite structure includes the metal substitute. In particular, it has been found that compressive loads above 25 MPa improves the antimicrobial properties of the fluoridated apatite structure and, further, that increasing the compressive load above 25 MPa further improves the antimicrobial properties of the fluoridated apatite structure.

The material that forms the fluoridated apatite structure of the scaffolds 100 disclosed herein includes fluoridated apatite that has been sintered at a temperature between about 950° C. and about 1,350° C., or more particularly between about 1,050° C. and about 1,250° C., or even more particularly about 1,100° C. and about 1,200° C. In a particular example, the fluoridated apatite structure of the scaffolds 100 may be sintered in air for 3 hours at 1250° C. with a heating and cooling rate of 2° C./minute, starting and finishing at room temperature. The inventors currently believe that fluoridated apatite sintered in the temperature range(s) disclosed above agglomerate to form a plurality of bonded agglomerations of fluoridated apatite that have a size, shape, and zeta potential that encourage adhesion between tissue cells and the fluoridated apatite (e.g., FA) in the scaffold.

In an unsintered state, fluoridated apatite (FA and/or FHA) particles exhibits a substantially rod-like or needle-like crystal structure. During sintering, the subject fluoridated apatite crystals agglomerate and exhibit various bulk structures and surface morphologies. For example, through sintering, the fluoridated apatite particles may be formed into agglomerates exhibiting greater three dimensional characteristics, such as substantially granular shapes (e.g., prismatic, pseudo-prismatic, rounded, spherical, semi-spherical, ellipsoid, or irregularly rounded shapes). The as-sintered fluoridated apatite particles may be substantially devoid of the rod-like or needle-like fluoridated apatite of the unsintered fluoridated apatite particles. The average volume of an average sintered fluoridated apatite agglomerate may be at least ten times the average volume of the average unsintered fluoridated apatite particles. The resulting sintered fluoridated apatite particles (e.g., agglomerates) exhibit an overall smoother surface morphology than the unsintered fluoridated apatite particles.

Bulk fluoridated apatite particles may be a coherent mass of agglomerations provided in a specific form, such as grains of the scaffold. Bulk fluoridated apatite particles may be formed by sintering a mass of fluoridated apatite particles and then grinding, crushing, or otherwise breaking the resulting sintered bulk body into smaller bulk particles. The smaller bulk particles may be sized, such as using a sieve, to provide a plurality of particles having a substantially homogenous average particle size. The bulk particle size (e.g., a coherent mass of agglomerations provided in a granular form) of the bulk fluoridated apatite particles disclosed herein may be at least about 5 μm, such as about 30 μm to 300 μm, about 60 μm to 200 μm, about 65 μm to 150 μm, about 60 μm to 120 μm, about 120 μm to 200 μm, or less than about 300 μm. The particles may be sieved to obtain the desired particle size.

The porosity of a scaffold of the sintered fluoridated apatite particles is also different than the porosity of a scaffold of the unsintered fluoridated apatite particles. For example, the bulk structure of the sintered fluoridated apatite particles exhibits less porosity than the unsintered fluoridated apatite particles. This is believed to be due to the agglomerates densifying (e.g., self-organizing or building into naturally fitting structures) during sintering, thereby providing less pore space therebetween than the unsintered particles.

Fluoridated apatite maintains a relatively strong mechanical strength, even after sintering. For example, fabricated heat-treated FA and FHA scaffolds, which showed enhanced osteoblast cellular adhesion and proliferation properties when compared to HA surfaces treated at the same temperatures, also showed compressive strengths of 100-200 MPa, which is similar to cortical bone (170-193 MPa cortical bone; 7-10 MPa cancellous bone).

The inventors currently believe that the porosity and surface morphology of the sintered FA particles increase adhesion to tissue cells (e.g., endothelial cells, osteoblasts, fibroblasts, etc.). The charge of the fluoridated apatite material also contributes to increased tissue adhesion. For example, the surface charge of the fluoridated apatite material is believed to increase differentiation of cells at the interface therebetween. The FA is more electronegative than FHA and HA. As shown below, experiments have demonstrated that sintered FA promotes adhesion to osteoblasts, and to a much higher degree than sintered FHA and HA.

The surface charge of the scaffold material may be measured as the zeta potential. In some cases, the zeta potential of FA is more than double the zeta potential of FHA or HA sintered under the same conditions. The zeta potential of the fluoridated apatite scaffolds disclosed herein may be less than (e.g., have a greater negative value than) about −10 mV, such as about −10 mV to −80 mV, about −20 mV to −65 mV, about −26 mV to −80 mV, about −26 mV to −65 mV, about −40 mV to −80 mV, less than about −26 mV, less than about −35 mV, or less than about −40 mV. The inventors currently believe that the electronegativity of the fluorine atoms in the fluoridated apatite drive the zeta potential lower and stimulate cell adhesion, such as by causing differentiation. The zeta potential of may be determined, for example, using a Massively Parallel Phase Analysis Light Scattering (MP-PALS) spectrometer.

Various techniques may be used to manufacture the implantable scaffolds disclosed herein. FIG. 2 is a flow diagram of a method 200 of making a scaffold, according to an embodiment. The method 200 includes block 210 of providing fluoridated apatite particles; block 220 of sintering the fluoridated apatite particles at a temperature of at least 950° C. to form a sintered body, and the block 230 of forming the implantable scaffold from the sintered body. In some embodiments, one or more of the blocks 210-230 may be omitted, combined with other blocks, or performed in a different order than presented. For example, the blocks 210 and 220 may be performed substantially simultaneously, such as via sintering.

Block 210 of providing fluoridated apatite particles may include providing FA particles, FHA particles, or combinations of the foregoing. Providing fluoridated apatite particles may include providing a plurality of fluoridated apatite particles, such as FA, FHA, or combinations thereof. In an embodiment, providing fluoridated apatite particles includes providing fluoridated apatite particles that include at least one metal substitute. The plurality of fluoridated apatite particles may exhibit any of the average fluoridated apatite particle sizes disclosed herein. The fluoridated apatite particles may exhibit any of the bulk fluoridated apatite particle sizes disclosed herein (e.g., 60 μm to 200 μm).

In some embodiments, providing fluoridated apatite particles may include forming the fluoridated apatite particles, such as FA particles, FHA particles, FA and/or FHA particles including the metal substitute, or mixtures thereof. In some embodiments, a precipitation (e.g., continuous aqueous precipitation) method may be used to synthesize the fluoridated apatite particles. In an example, the precipitation method may include substituting at least some of the calcium with the metal substitute. In an example, forming the fluoridated apatite particles includes pre-sintering the fluoridated apatite particles.

In a particular example, forming the fluoridated apatite particles includes forming fluoridate apatite particles having zinc substituted therein. In such an example, forming the fluoridated apatites particles may include using a calcium source (e.g., Ca(NO₃)₂.H₂O), a zinc source (e.g., Zn(NO₃)₂), a phosphate source (e.g., Na₂HPO₄), and a fluorine source (e.g., NaF). To synthesize the fluoridated apatite particles, the calcium, zinc, phosphate, and fluorine sources may be mixed at boiling point in stoichiometric ion solutions of Ca²⁺, PO₄³⁻, and F⁻ (CA²⁺/PO₄³⁻/F⁻=5:3:1 molar ratio). For x molar % zinc-fluoridated apatite particles syntheses, x molar % Ca²⁺ cation solution may be replaced by x molar % Zn²⁺ ions to maintain the molar ratios of the reaction to be (Ca²⁺ Zn²⁺)/PO³⁻/F⁻=5:3:1. It is noted that this specific method of forming the fluoridated apatite particles may include a metal substitute source other than or in addition to zinc.

In some embodiments, providing fluoridated apatite particles may include forming the plurality of fluoridated apatite particles into a coherent body. The coherent body may consist of or consist essentially of FA, FHA, one or more stromal vascular fraction, one or more dopants, or combinations of any of the foregoing. In some embodiments, additional materials may be present in the coherent body, such as a ceramic, metal, polymer, etc. Forming the plurality of fluoridated apatite particles into a coherent body may include pressing, rolling, molding, casting (e.g., foam casting), adhering, three-dimensional printing, or otherwise forming an at least partially bonded body or mass of fluoridated apatite particles. In an example, forming the plurality of fluoridated apatite particles into a coherent body includes pressing the fluoridated apatite particles at any of the compressive loads disclosed above. In an example, the coherent body may be created by forming a slurry having fluoridated apatite particles and a sacrificial structural material. The slurry may be dried, cooled, or reacted to harden into the coherent body. The coherent body includes a solid or semi-solid structure containing fluoridated apatite particles and the sacrificial structural material (e.g., investment material). The coherent body may be frozen or compressed to form a green state part that remains intact as a solid unitary structure. The sacrificial structural material may be selected to harden at a desired temperature or condition (800° C.-1300° C.), to provide a selected porosity, and/or to be removable from the coherent body (e.g., plurality of at least partially bonded fluoridated apatite particles) via one or more of combustion, melting, dissolution, vacuum, or any other technique for removing an investment material. For example, the sacrificial structural material may be a polymer, a salt, a ceramic, or the like, composed to dissolve or otherwise dissociate in selected conditions. The sacrificial structural material may be removed prior to, after, or concurrently with sintering the fluoridated apatite particles using any of the sintering techniques disclosed herein.

In an example, forming the plurality of fluoridated apatite particles into a coherent body includes foam-casting the particles. In such an example, aqueous slurries may be made. The slurries may include binders (e.g., 1 wt % polyvinyl alcohol and 1 wt % polyethylene glycol), a dispersant (e.g., 1 wt % Dynol 604), and distilled water. The slurries may infiltrate and fill a correctly sized foam template and dried under vacuum. The resultant green bodies may then be heated (e.g., sintered) to remove the foam template.

In some embodiments, the porous scaffoldings may be fabricated out of a fluoridated apatite slurry. The fluoridated apatite (e.g., FA) slurry may be used as an investment material or casting material. For example, scaffolds may be prepared using polymeric sponges as investment material or a mold, which are then infiltrated with the fluoridated apatite slurry containing monomers and initiators for rapid gelation via in situ polymerization. This gel sponge processing technique integrates gel-casting with polymer sponge methods. Next, the polymeric sponge can be removed (e.g., burned off at elevated temperatures (e.g., 1,050° C. to 1,250° C.)) and the remaining coherent body (e.g., fluoridated apatite scaffold) may be cleaned with distilled water.

In some embodiments, providing fluoridated apatite particles may include forming the fluoridated apatite particles into a predetermined shape. For example, fluoridated apatite scaffolding with pre-determined shapes (e.g., flat, tubular, or cubic) and porosities. Fluoridated apatite particles, DI water, and a binder (e.g., acrylamide/methylenebysacrylamide) may be mixed such as in a ball-mill and then, one or more of an initiator (e.g., Tetramethylenediamine), binder (e.g., carboxymethyl cellulose or Polyvinyl alcohol), dispersant, surfactant, or excess DI water may be added and mixed for duration (e.g., 12 hours). This slurry may be cured under vacuum, and sequentially poured over an infiltrated into a shaped polyether sponge as a frame for obtaining the desired shape, size, and porosity. The infiltrated sponge may be put under vacuum and a catalyst (e.g., ammonium persulfate) solution may be applied for facilitating polymerization. The sponges may be placed inside a nitrogen chamber to avoid surface contamination, which may prevent the polymerization process. After drying at room temperature, samples may be sintered to form the sintered body as disclosed in more detail below (e.g., at 1250° C. at a heating rate of 5° C./min).

Further methods of forming a coherent body of fluoridated apatite particles may include mixing the fluoridated apatite particles with polymers, slip casting, freeze-casting, sol-gel formation, foaming, polymer replication, solid freeform fabrication, three-dimensional printing, or the like.

The fluoridated apatite particles in the coherent body may be further subjected to sintering at a predetermined temperature. Block 220 of sintering the fluoridated apatite particles to form a sintered body may include sintering the fluoridated apatite particles prior to, contemporaneously with, or after providing the fluoridated apatite particles. The sintered body may have a denser bulk structure than the unsintered coherent body. The porosity of the sintered body may exhibit less porosity than the unsintered coherent body. Sintering the fluoridated apatite particles may include sintering the coherent body. Sintering the fluoridated apatite particles may include sintering a coherent body of fluoridated apatite particles that have been previously sintered. The sintered body may consist of or consist essentially of FA, FHA, one or more dopants, or combinations of any of the foregoing.

The fluoridated apatite particles may be sintered as one or both of a loose powder or in the cohesive body (e.g., polymer sponge impregnated with FA particles or pressed pellet of subject FA particles). Sintering the fluoridated apatite particles may include heating the fluoridated apatite particles to a temperature of at least about 950° C., such as about 950° C. to about 1,350° C., about 1,050° C. to about 1,250° C., about 1,050° C. to about 1,150° C., about 1,150° C. to about 1,250° C., at least 1,050° C., at least about 1,150° C., less than about 1,300° C., or less than about 1,250° C. The heating (e.g., sintering) may be carried out for at least 1 minute, such as about 1 minute to about 24 hours, about 1 hour to about 18 hours, about 2 hours to about 12 hours, about 4 hours to about 10 hours, about 20 minutes to about 4 hours, about 30 minutes to about 3 hours, about 1 hour to about 10 hours, about 8 hours to about 16 hours, at least about 2 hours, less than about 24 hours, or less than about 12 hours. The above-noted sintering times may be hold times at the sintering temperature. For example, a plurality of fluoridated apatite particles may be placed in a sintering oven that is ramped up to the sintering temperature at a selected rate (e.g., about 5° C./min., about 7° C./min., about 10° C./min., about 5° C./min. to 15° C./min, or about 1° C./min or more), maintains the sintering temperature for the selected duration, and ramps back down to the ambient temperature at a selected rate (e.g., any of the rates disclosed above). The sintering temperatures within the ranges disclosed herein do not alter the chemical composition of the fluoridated apatites disclosed herein. Sintering may be carried out in an inert environment or an ambient environment.

Sintering the fluoridated apatite particles may include heating the fluoridated apatite particles in an inert atmosphere (e.g., N₂ or Argon), in a vacuum, in an open or oxidizing atmosphere (e.g., in the presence of oxygen, carbon dioxide, N₂, etc.), or combinations of any of the foregoing.

Sintering the fluoridated apatite particles to form a sintered body may include sintering the fluoridated apatite particles (e.g., coherent body) at a temperature sufficient to burn out any investment or mold material such as a polymer, so that substantially only the fluoridated apatite or other selected materials desired for implantation remain. In such embodiments, the as-cast fluoridated apatite particles (e.g., coherent body) and the material the fluoridated apatite particles were cast in (e.g., polymer sponge or matrix material) may be subjected to sintering.

The sintered fluoridated apatite particles may exhibit the surface morphology, porosity, zeta potential, average particle size, or any other characteristics of any of the sintered fluoridated apatite particles disclosed herein. For example, the sintered body may include sintered fluoridated apatite particles having a spherical, semi-spherical, prismatic, pseudo-prismatic, ellipsoid, or irregularly rounded shape and are devoid of rod-like or needle-like fluoridated apatite particles.

The sintered fluoridated apatite particles are densified via the sintering process while the polymer material is combusted or melts out of the coherent body. The shrinkage of the fluoridated apatite particles during sintering is reproducible (about 15% upon sintering). Accordingly, the techniques disclosed herein provide the ability to custom make scaffoldings for the desired shapes and sizes to fit the clinical needs of grafts for reconstructive surgeries in plastic, orthopedic and dental surgeries. Unfortunately, tensile properties of pure apatite ceramics are limited. For example, unsintered apatite ceramics exhibit a hardness value about of 5.1 GPa and fracture toughness value of about 2.0 MPa·m^{1/2}. When compared to the fracture toughness of human bone (about 12 MPa·m^{1/2}), apatite's toughness is relatively poor. Thus, the fluoridated apatite scaffolds disclosed herein may be used as heavy-loaded implants after sintering to improve strength.

The scaffolds disclosed herein (e.g., FA scaffolds) may exhibit at least 10% porosity, such as 30% to 70% porosity. Accordingly, the scaffolds may provide a ready delivery means for one or more dopants. Such scaffolds may be used as bone fillers, such as for dental applications or the like.

Returning to FIG. 2, the block 230 of forming the scaffold from the sintered body may include shaping or sizing the sintered body into a selected shape and size. For example, the sintered body may be shaped and sized to fit a pocket in tissue of a subject. Forming the scaffold from the sintered body may include machining, grinding, lasing, carving, polishing, lapping, or otherwise removing material from the sintered body. The scaffold may be sized and shaped as a percutaneous implant, an osseointegrated implant, a dental implant, a bone implant, bone replacement, or the like.

In some embodiments, forming the scaffold may be carried out substantially simultaneously with sintering the coherent body to form a sintered body. For example, the forming the scaffold and sintering the coherent body may both be carried out via sintering. In such embodiments, the coherent body may be provided in a size and shape such that the sintered body may be the scaffold (e.g., implantable size and shape). Providing such a shape may be provided or formed by one or more of molding, grinding, cutting, lapping, etc.

In an embodiment, when the integration aid includes a stromal vascular fraction, the method 200 may include disposing the stromal vascular fraction in or on the fluoridated apatite particles, coherent body, sintered body, or scaffold with the stromal vascular fraction. In an example, the method 200 may include obtaining adipose tissue from the subject and isolating the stromal vascular fraction from the adipose tissue. The stromal vascular fraction obtained from the adipose tissue may then be disposed in or on the fluoridated apatite structure. In a particular example, the method 200 may include obtaining adipose tissue from the subject and implanting the implantable scaffold in the subject during the same procedure. In an example, the method 200 may include providing non-adipose stromal vascular fraction and disposing the non-adipose stromal vascular fraction in or on the fluoridated apatite structure. In an example, the stromal vascular fraction may be disposed in or on the fluoridated apatite structure after sintering the fluoridated apatite structure since sintering the stromal vascular fraction may damage the stromal vascular fraction.

In an example, the stromal vascular fraction may be provided in a liquid form exhibiting a viscosity sufficient to allow the cells to be disposed in and/or on the fluoridated apatite structure. In an example, disposing the stromal vascular fraction in and/or on the fluoridated apatite structure may include a solution containing the stromal vascular fraction (i.e., diluted stromal vascular fraction) cells into or onto the coherent body, sintered body, or scaffold. For example, the stromal vascular fraction may be suspended, dispersed, or dissolved in a liquid medium which is applied to the coherent body, sintered body, or scaffold, such as via immersing, spraying, pipetting, aliquoting, pouring, or any other liquid application technique. In embodiments, a scaffold may be wetted and loaded with a suspension of stromal vascular fraction.

The method 200 may include disposing sufficient quantities of stem cells within the stromal vascular fraction that can stimulate bone growth, cell differentiation, or vascularization. For example, the stromal vascular fraction may be present in and/or on the fluoridated apatite structure in cell densities 10² cells/ml to 10¹⁰ cells/ml per unit area, such as in ranges of 10² cells/ml to 10⁴ cells/ml, 10³ cells/ml to 10⁵ cells/ml, 10⁴ cells/ml to 10⁶ cells/ml, 10⁵ cells/ml to 10⁷ cells/ml, 10⁶ cells/ml to 10⁸ cells/ml, 10⁷ cells/ml to 10⁹ cells/ml, or 10⁸ cells/ml to 10¹⁰ cells/ml. The amount of cells present in stromal vascular fraction of the implantable scaffold may be selected based on the size of the implantation site and the size of the implantable scaffold.

The method 200 may include doping the fluoridated apatite particles, coherent body, sintered body, or scaffold with one or more dopants, such as any of the dopants disclosed herein. Doping the fluoridated apatite particles may include mixing one or more dopants into the fluoridated apatite particles prior to, contemporaneously with, or after providing the fluoridated apatite particles or forming the coherent body of fluoridated apatite particles. For example, doping the fluoridated apatite particles may include adding one or more dopants to the plurality of fluoridated apatite particles prior to forming the coherent body, or coating at least a portion of the coherent body with one or more dopants after forming the coherent body. Each of the one or more dopants may be present in amounts composed to stimulate bone growth, cell differentiation, or soft tissue growth, such as at least 1 nanogram (ng), 10 micrograms (g) to 10 milligrams (mg), about m to 1 mg, 50 μg to 500 μg, or less than 1 mg.

Combinations of dopants may be utilized to provide controlled release of one or more dopants in vivo. For example, ADSCs may be used with BMP-2 and a hydrogel such as keratose, where keratose is relatively stable in vivo to allow for controlled release of dopants disposed therein. For example, the keratose may be applied as a coating over the scaffold, where upon degradation of the keratose, the dopants there beneath are released. For example, the keratose may dissolve to release growth factors such as BMP-2. In some examples, the dopants may be present in a layered systems where multiple layers of dopants are each disposed beneath a layer of hydrogel such as keratose. Accordingly, time released benefits may be realized using the scaffolds disclosed herein.

Doping the coherent body, sintered body, or scaffold may include applying a solution containing the one or more dopants into or onto the coherent body, sintered body, or scaffold. For example, one or more of the dopants may be suspended, dispersed, or dissolved in a liquid medium which is applied to the coherent body, sintered body, or scaffold, such as via immersing, spraying, pipetting, aliquoting, or any other liquid application technique. In embodiments, BMP-2 may be dispersed in a keratose hydrogel, which may be poured over a scaffold and allowed to incubate. Similarly, a scaffold may be wetted and loaded with a suspension of ADSCs.

In some examples, doping the scaffold may include disposing the scaffold in the tissue of an implantee, such as soft tissue to deposit autologous tissues, growth factors, etc. in the scaffold prior to final implantation in a bone.

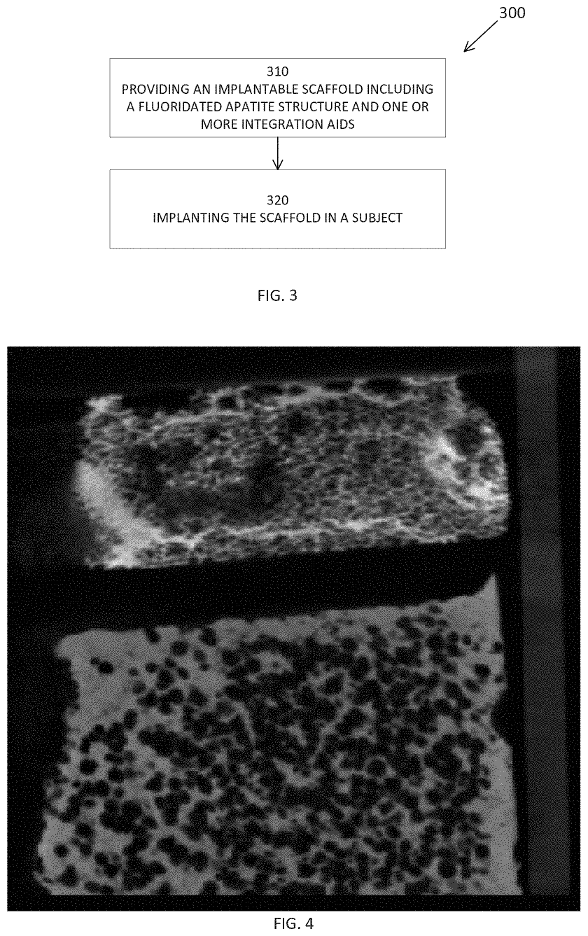

FIG. 3 is a flow chart of a method 300 of using a scaffold having fluoridated apatite, according to an embodiment. The method 300 includes the block 310 of providing an implantable scaffold including a fluoridated apatite structure and one or more integration aids and the block 320 of implanting the scaffold in a subject.

The block 320 of providing an implantable scaffold including fluoridated apatite structure and one or more integration aids may include providing any of the scaffolds disclosed herein. Providing the implantable scaffold may include providing an implantable scaffold having fluoridated apatite structure sintered at any of the temperatures disclosed herein (about 950° C. to about 1350° C. or about 1050° C. to about 1250° C.), having any of the zeta values disclosed herein, having any of the surface morphologies disclosed herein, or any of the properties of sintered fluoridated apatite particles disclosed herein. The implantable scaffold may consist of or consist essentially of FA, FHA, one or more integration aids, or combinations of any of the foregoing. The implantable scaffold may be sized and shaped for at least partial bone replacement, an osseointegrated implant, a dental implant, or the like. Providing the implantable scaffold may include making at least a portion of the implantable scaffold, such as by using any of the techniques for making scaffolds disclosed herein. For example, making at least a portion of the implantable scaffold may include isolating stromal vascular fraction from adipose tissue.

The block 320 of implanting the implantable scaffold in a subject may include implanting the implantable scaffold into the tissue of a subject, such as into the skin, bone, or other tissues of a subject. For example, implanting the implantable scaffold in a subject may include positioning the implantable scaffold within a pocket in a bone of a subject, such as in a jaw, hip, vertebrae, femur, etc. For example, an osseointegrated scaffold may be inserted into bone whereby the fluoridated apatite structure contacts one or both of the bone or soft tissue of the subject. Implanting the implantable scaffold in a subject may include surgically implanting the scaffold into the tissue of a subject. Implanting the scaffold in a subject may include closing the implantation site, such as by suturing.

Implanting the scaffold may include one or more of sizing or shaping the implantable scaffold as disclosed herein.

The method 300 may include preparing an implantation site such as by removing tissue (e.g., bone) from an implantation site in a patient. For example, preparing an implantation size may include removing at least some bone to form a pocket in a bone. In such embodiments, the scaffold may be shaped and sized to fit in the pocket. Preparing the implantation site may also include removing tissue and then isolating stromal vascular fraction from the removed tissue.

Preparing the implantation site may include cleaning the implantation site, such as cleaning the interface between the bone of the subject and the scaffold. Such cleaning may include washing with water or another fluid (e.g., iodine, soap, alcohol, etc.).

The method 300 may include implanting the implantable scaffold in soft tissue prior to implanting the implantable scaffold in bone, such as to allow the one or more dopants to produce autograft cells. The implantable scaffold may be disposed in the soft tissue for at least a week, such as 1 week to 3 months.

In a particular example, the method 300 may include anesthetizing the subject until intubated. The skin around the incision may be shaved, prepared, and draped for sterile surgery. Initially, skin and subcutaneous incisions may be made. Adipose tissue in this region may be collected and placed in a sterile lactated Ringer solution. The collected adipose tissue may be weighed, rinsed with lactated Ringer solution, suspended in collagenase solution for a time period (e.g., 30 minutes), centrifuged, and then the stromal vascular fraction may be collected. The viable cells in the stromal vascular fraction may then be determined using a small sample of the stromal vascular fraction (e.g., staining with DAPI and counting the number of nucleated cells with an automated cell counter). The implantable scaffold may then be placed in a concentrated solution of the stromal vascular fraction (e.g., 1×10⁷ stromal vascular fraction cells in a Ringer solution) for a define time period sufficient to allow adherence of cells on the implantable scaffold. The implantable scaffold may then at least a portion fill a bone defect. After placing the implantable scaffold, the tissue cut during the incision may then be closed.

Further examples of implantable structures, methods of making implantable structures, and methods of using implantable structures that may be used in any of the embodiments disclosed herein are disclosed in U.S. patent application Ser. No. 17/420,579 filed on Jul. 2, 2021 and U.S. patent application Ser. No. 17/420,589 filed on Jul. 2, 2021, the disclosure of each of which is incorporated herein, in its entirety, by this reference.

### WORKING EXAMPLES

The following working examples provide further detail in connection with the specific embodiments described above.

### Characterization of Scaffolds

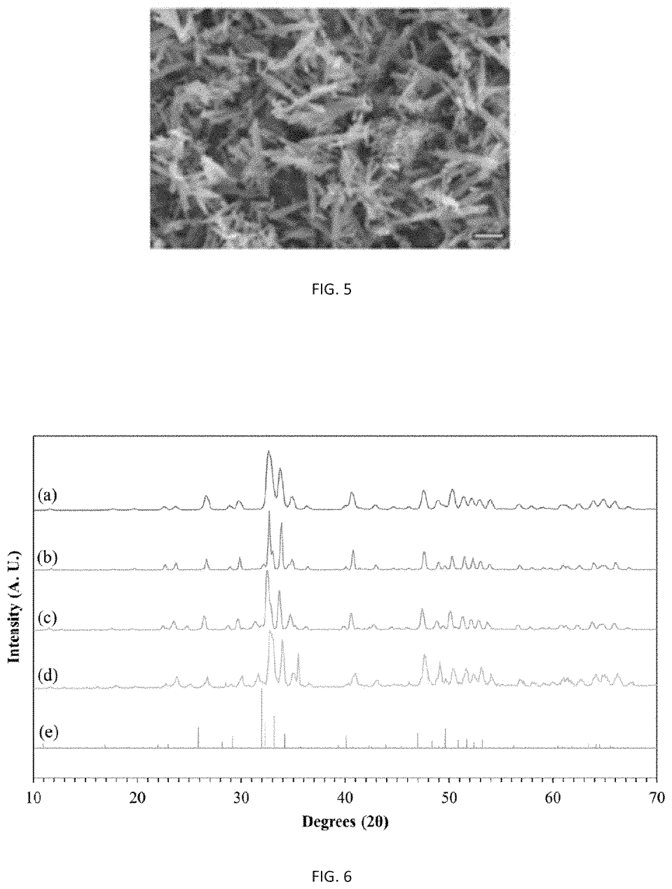

Several implantable scaffolds formed from HA and FHA were manufactured. FIG. 4 is a micro-CT image of the porous structure of an FA material (top) and a HA material (bottom). FIG. 5 is a scanning electron microscopy image of an FHA material as synthesized. —Although it is important to improve the mechanical strength, it is equally important to maintain the chemical composition and crystallinity of the fluoridated apatite structure. At high temperatures, it is known that apatite can undergo phase changes to tricalcium phosphate structures. Therefore, to confirm that the chemical compositions and crystallinities of the sintered powders were maintained, sintered powders were subjected to x-ray diffraction (XRD) studies. FIG. 6 illustrates X-ray diffraction patterns of (1) as-made FA powder, (b) FA sintered at 1250° C., (c) FA sintered at 1350° C., (d) FA sintered at 1450° C., and (e) an HA reference pattern. The peak shifts observed compared to the HA reference pattern reflect the change in lattice parameters of the apatite structures resulting from fluorine incorporation. FA sintered at 1250° C., and 1350° C. showed a narrowing of peaks compared to the unsintered FA, indicating an increase in crystallinity with sintering temperature up to temperatures of 1350° C. A combination of peak broadening or amorphous humps appeared more prevalent for FA sintered at 1450° C., indicating a phase-change event. Peak broadening was attributed to small crystallite size, while amorphous structures would be due to phase decomposition into β-tricalcium phosphate (β-TCP). Overall, the XRD analysis corroborated the increased thermal and chemical stability of the sintered FA up to 1350° C. Collectively, above data showed that the sintering temperature below 1350° C. will maintain the chemical structure and composition of the apatites while providing adequate mechanical strength similar to the human trabecular bone. Typically, the modulus of human trabecular bone ranges between 10 and 3,000 MPa.

### Scaffold Manufacturing Using Foam-Casting Techniques

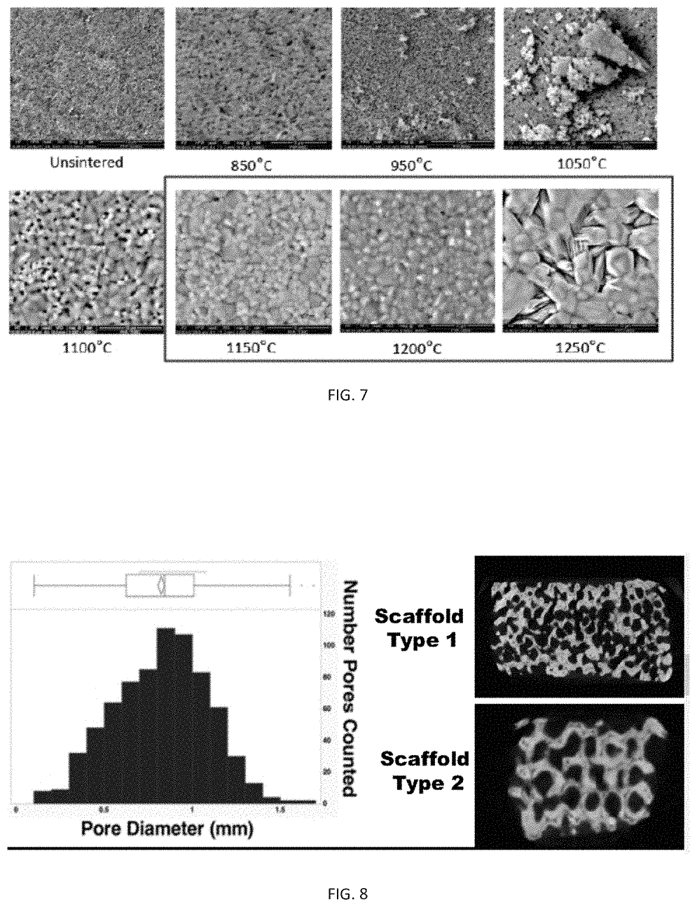

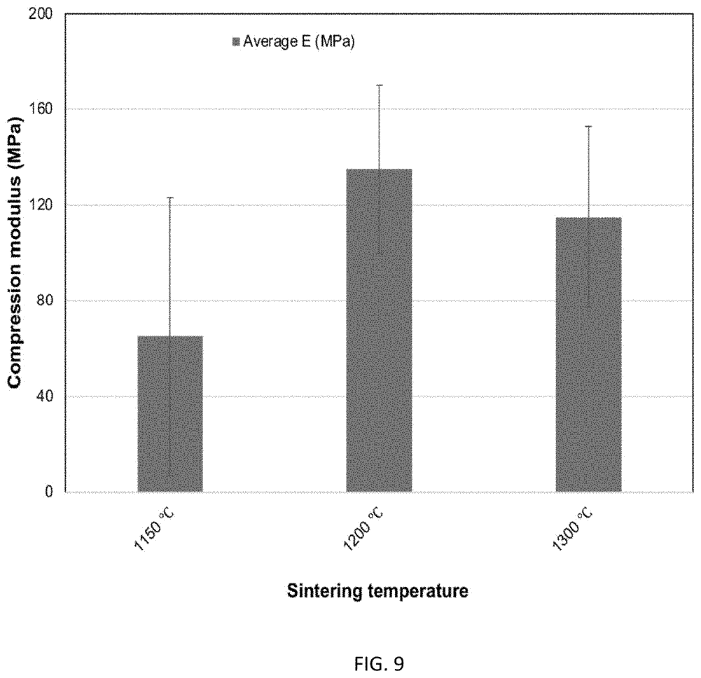

Implantable scaffolds including FA were fabricated using a foam-casting technique and then sintered at a range of temperatures between 850° C. and 1450 C°. FIG. 7 is a set of SEM images of the implantable scaffolds before sintering and after sintering at various temperatures, showing the formation of micro-structured surface topography between 1150 and 1200° C. Whether the image is of an unsintered FA sample or a sintered FA scaffold is shown in FIG. 7. The images within the box shown in FIG. 7 demonstrates that the formation of micro-crystallites is possible at high temperatures corroborating the XRD data of FIG. 6. The sintered scaffolds were then imaged using micro-CT. FIG. 8 is a representative histogram (left) of the pore size distributions for five foam-casted porous scaffolds and representative images of micro-CT scans (right) used to generate the histogram. FIG. 8 demonstrates that the foam-casted porous scaffolds exhibited a mean pore size of 820±270 μm. The 2D micro-CT slices visually depict the interconnected open porous structure of the scaffolds in both the longitudinal view and the cross-section view of the scaffolds. In particular, FIG. 8 illustrates that the sintered scaffolds had interconnecting pores of various sizes. Quantitatively, the porosity ranged between 45% to 60% and pore size ranged between 100 to 1500 μm. It is believed that scaffolds exhibiting these porosity ranges and pore size ranges are ideal for both promoting bone ingrowth and the differentiation of cells within the stromal vascular fraction. Post-characterization, uniaxial compression strengths were calculated using a universal tensiometer. FIG. 9 is a graph illustrating the calculated compression strengths for several foam-casted scaffolds sintered at various temperatures. FIG. 9 shows that the compression strength of the foam-casted sintered FA scaffolds is comparable to human cancellous bone tissues (10-500 MPa).

### In Vitro Assays Including Stromal Vascular Fraction

An ideal engineered bone implant should replicate the beneficial qualities of autograft bone, including structural support, a reliable source of osteogenic cells, and the capacity to generate osteogenic signals. Since apatites are known for their osteoconductivity and, as presented above, higher sintering temperatures result in mechanically robust scaffolds, it is vital to include at least one integration aid to make them an effective autograft-like scaffold. As stated above, the integration aid may include stromal vascular fraction. To confirm the effectiveness of fluoridated apatite structure to guide the differentiation of stromal vascular fraction, in vitro studies were carried out. The stromal vascular fraction is separated from adipose (fat) tissue. Stromal vascular fraction comprised pervascular cells, leukocytes, endothelial cells, other adipose cells, fibroblasts, and progenitor stem cells. The stromal vascular fraction was extracted from the subject's own body and administered to effect therapeutic results. As stromal vascular fraction can differentiate into multiple lineages, it was important to access the fluoridated apatite structure ability to differentiate stromal vascular fraction to the osteogenic pathway.

First, it was determined the impact that HA, FA, FHA, and sintering temperatures have on the viability of cells within stromal vascular fraction and fluoridated apatites' ability to differentiate into the osteoblast lineage. For this, small HA and fluoridated apatite structure disks were prepared from the raw powder. These disks were sintered at pre-selected temperatures. Post-sintering, a scanning electron image was used to confirm the micro-scaled topographical features of the surface of the small HA and fluoridated apatite structures. A known density (1,300 cells/cm²) of passage 3-5 ADSCs (RASMD-01001, Santa Clara, Calif.) were incubated on the disks at 37° C. in a 5% CO₂ incubator for 2 and 10 days. Cells were mechanically detached from the surface at 2 and 10 days, post-seeding, and subjected to cell viability assays (alamarBlue® assay), immunohistochemistry, and RT-PCR analysis, data is compared to cells plated onto a cell culture dish (cell drop controls).

At two days post-seeding, it was found that were relatively more viable cells on the HA 1150° C. surface (1.9±0.09) compared to all other groups, suggesting stromal vascular fraction either preferentially proliferated or selectively adhered to a greater extent on this surface (p<0.05; data not given). Additionally, the cell drop control group had statistically fewer viable cells than the HA, FA, and FHA apatite disks sintered at 1250° C. By 10 days post-seeding, there were no statistical differences between the cell drop control group and the different apatite surfaces (p>0.05), indicating all of these surfaces support the cell adhesion and growth.

For assessing these surfaces' ability to promote differentiation, total RNAs were extracted using standard techniques. After confirming the quality, RNAs were reverse transcribed. Gene expression was then quantified using real-time PCR with gene-specific primers for runt-related transcription factor 2 (Runx2; RN01512298_m1; NM_001278483.1) and secreted phosphoprotein 1 (SPP1, also known as osteopontin; RN00681031_m1, Thermo Fisher ID; NM_012881.2, NCBI Reference Sequence). All values were normalized to the housekeeping gene 18s ribosomal RNA (Hs999999901_s1; X03205.1, GenBank) and data reported as 2\hat{ }ΔΔct and calculated relative to the cell drop control. To confirm the mRNA expression data, ADSCs seeded on surfaces for 2 or 10 days were fixed in 10% formalin and then incubated with a primary antibody for osteocalcin or osteopontin. The samples were then incubated with fluorescently labeled secondary antibodies for osteocalcin and osteopontin and were imaged using a confocal microscope at 10× magnification.

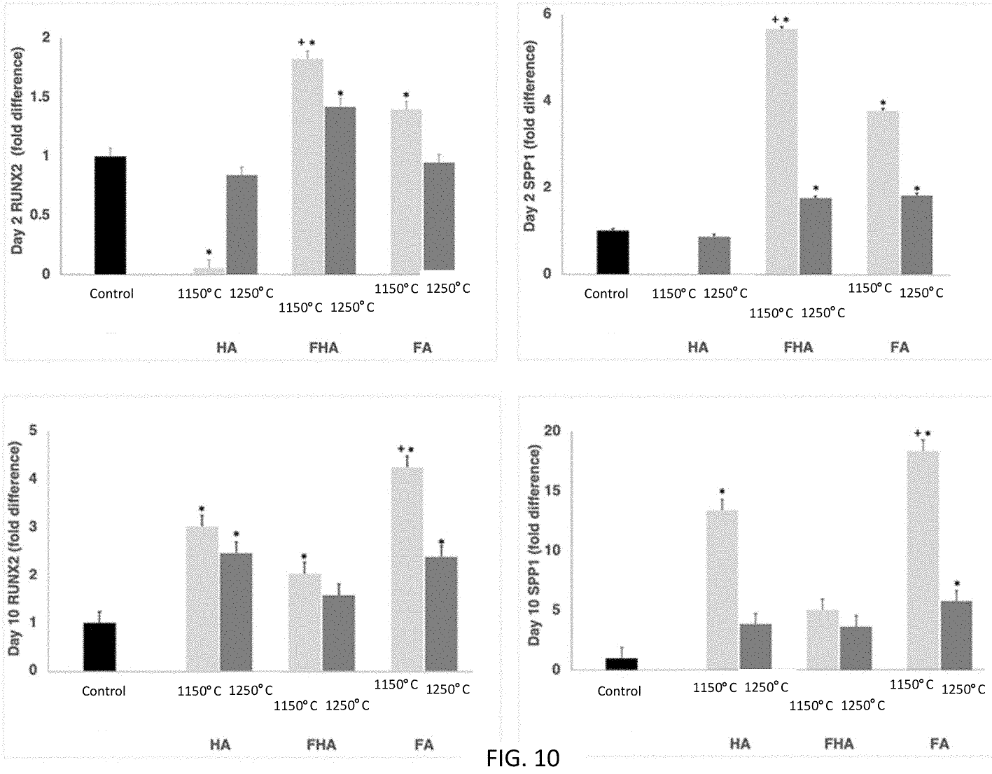

RUNX2, a transcription factor associated with the early differentiation of stem cells into pre-osteoblast cell lineage, was expressed at lower levels in ADSCs plated on HA1150° C. (p<0.01) and at equivalent levels to the cell drop control on HA1250° C. This data are supported by the expression of SPP1—a marker of late-stage osteoblast differentiation which was undetectable in cells plated on HA sintered at 1150° C. and equivalent when compared to the cell drop control plated on HA1250° C. FIG. 10 is a graph illustrating the days 2 and 10 RT-PCR, mRNA expression data for osteogenic markers, Runx2 (left) and SPP1 (right). Taken together, the data shown in FIG. 10 suggest that, at two days post-seeding, HA sintered at 1150° C. and HA sintered at 1250° C. did not promote extensive differentiation of the stromal vascular fraction into bone lineage cells to a greater degree than cells plated onto a cell culture dish (cell drop control). In contrast with the HA groups, there was greater expression of the early osteoblast differentiation marker RUNX2 in cells grown on FA sintered at 1150° C. and FHA sintered at 1150° C. surfaces compared to the cell drop control (p<0.05). In addition, there was significantly more RUNX2 expression in cells seeded on the FHA sintered at 1250° C. group. However, there was no difference noted in the expression of RUNX2 between ADSCs seeded on FA sintered 1250° C. and the cell drop control. Also, the levels of expression of SPP1 mRNA, another marker of osteoblast differentiation, were elevated when stromal vascular fractions were seeded onto FA sintered at 1150° C., FA sintered at 1250° C., FHA sintered at 1150° C., and FHA sintered at '1250° C. and were statistically different (p<0.01) than the cell drop control. By day 10, on average, the osteoblast markers Runx2 and SPP1 were expressed in the stromal vascular fraction cell plated on FA and FHA to a greater degree than those plated on the cell drop control surface. At 10 days, although all apatite surfaces appeared to favor the osteogenic pathways, late osteogenic marker SSP1 was overexpressed in FA sintered at 1150° C., and this was moved forward to the pilot rat study as the candidate surface type.

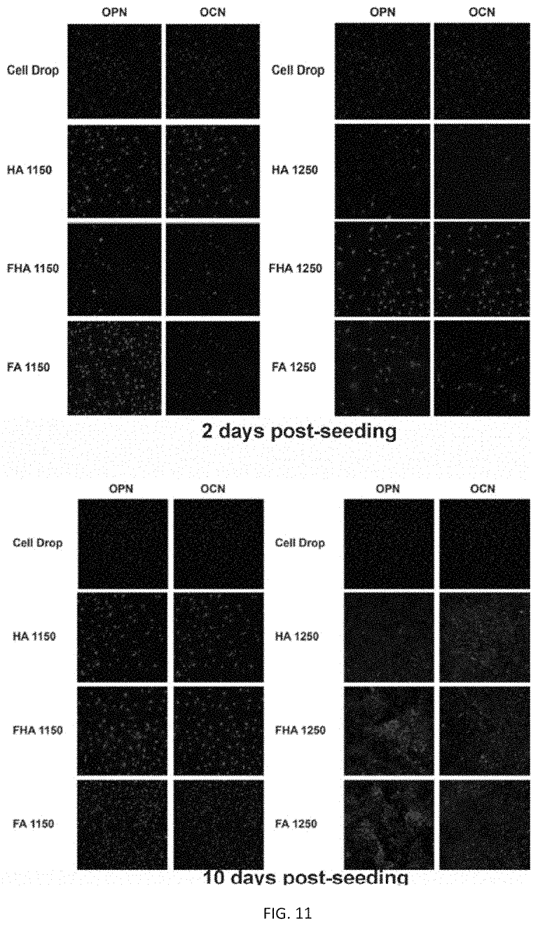

FIG. 11 includes images showing the osteoblast markers expressed at 2 and 10 days post-seeding. On day 2, cells growing on FA and FHA stained positive for both osteopontin (OPN, encoded by the SPP1 gene) and osteocalcin (OCN), while at this time point there was very little or no staining for both proteins on the HA surfaces and the cell drop control. This is consistent with the PCR data. At day 10, all bone surfaces and sintering temperatures expressed higher levels of both osteopontin and osteocalcin protein than the cell drop control. The combined PCR and immunohistochemistry data suggest that stromal vascular fraction had transformed into an osteoblast lineage earlier when they were seeded onto FA and FHA surfaces than when they were seeded onto HA surfaces. This is an important finding since surfaces themselves drive the stromal vascular fraction to the osteogenic pathway without any external stimuli, such as growth factors or cyclic stresses/strains. It is noted that FIG. 11 illustrates that the stromal vascular fraction grown on FA and FHA that were sintered at 1150° C. and 1250° C. appeared to having a greater expression of the osteoblast markers OPN and OCN when compared to the expression of the same markers when stromal vascular fractions were grown on HA and cell drop control. Additionally, there is more staining on the 1150° C. surfaces than the 1250° C. surfaces. Further, FIG. 11 illustrates that, for 10 days post-seeding, there were greater expressions of OCN and OPN on surfaces sintered at 1250° C.

Next, SVF cells were harvested and characterized from humans and rats. For determining the composition of SVFs from rats, the abdominal fat from 5 male Lewis rats were gathered and weighed. These fat samples were then digested with collagenases (1 and 11) for one hour at 37° C., the red blood cells lysed, and the remaining cells were suspended in media. After lysing the red blood cells, finally detached cells were washed and suspended in media. A small sample of the cell suspension was stained with DAPI and evaluated along with AccuCount beads by fluorescent activated cell sorting (FACS) to determine the concentration of viable cells within our samples. On average, 8500±2300 cells/gram of fat were obtained. This experiment was repeated for human fat, which was harvested using the approved protocol (IRB:1094 Molecular Classification of Cancer). A higher concentration of cells was obtained from human tissues (125,000±28,000 cells/gram of fat). In order to quantify the number of ADSC cells within the SVF portion, these cells were labeled with CD45, CD31, CD34, CD 73, and CD 90, stained for DAPI, and then sorted using FACS. Here, we defined stem cells as viable cells, negative for CD45 and CD31, and positive for CD34, CD 73, and CD 90. On average, ˜68±4% cells were alive within our extracted suspension and of those live cells, about 22% were stem cells (i.e., ADSC). It is important to note that all samples had a similar amount of viable cells post-processing. However, the number of ADSC cells differed from human fat sample to sample.

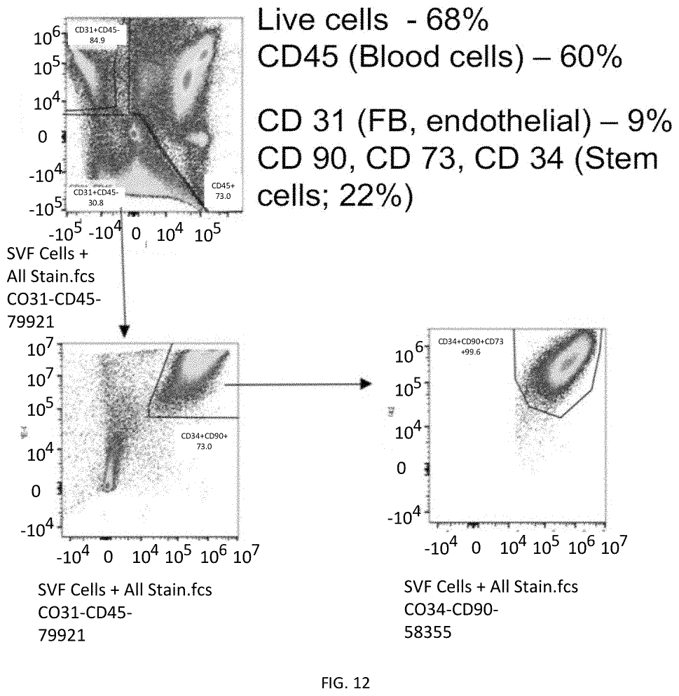

FIG. 12 is a representative set of fluorescence-activated cell sorting data of the freshly harvested human stromal vascular fraction cells. The majority of the cells were CD 45 positive blood cells. Within the live cell population, approximately 22% were CD 34 positive cells, which co-expressed CD 73 and CD90 representing the stromal vascular fraction cell population.

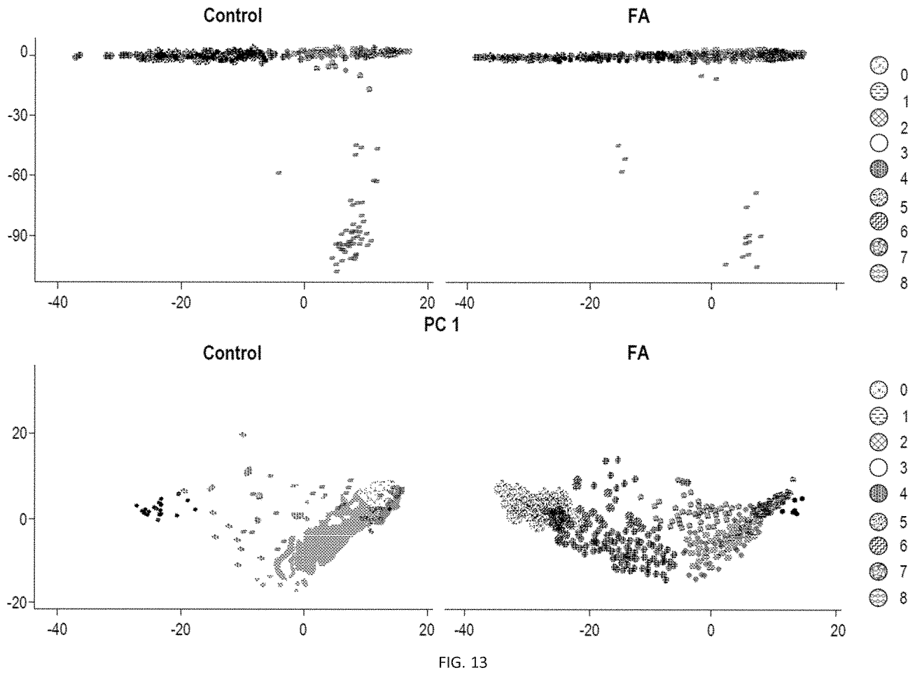

Next, a transcriptome study was undertaken to compare the differential expression of transcriptomes of the stromal vascular fraction cells grown on sintered FA surfaces versus cell culture plate control. For this, the stromal vascular fraction cells isolated from human fat were seeded on cell culture plate wells (control) and FA disks at a seeding density of 80,000 cells/1.9 cm². Growth media was selected instead of differentiating media in order to limit the media-induced differentiation of cells. After culturing for 6 days, samples were pooled, and scRNA-sequencing was performed at the University of Utah's genomics core. CellRanger and Seurat analysis packages were used for sequence alignment, quality control, feature quantification, and clustering, while SingleR was used for cell-type annotation based on the cell. FIG. 13 is a graph illustrating the principal component (PC) analysis of stromal vascular fraction cells grown on a control surface (cell culture flask) and FA surface, post 11 days of culturing. FIG. 13 shows that cell clusters were identified among the cultured human stromal vascular fraction cells (control) and stromal vascular fraction grown on FA selectively adhered to and proliferated on the FA surface. PC1 vs. PC2 identified the most spatially variable cluster 8, which was identified as adherent immune cells. PC1 vs. PC3 further separated less spatially clustered cells. Since the cells are inferred based on the expression level in the analysis package, a panel of phase-specific marker genes (G2/M and S) is needed to remove the cell cycle effects.

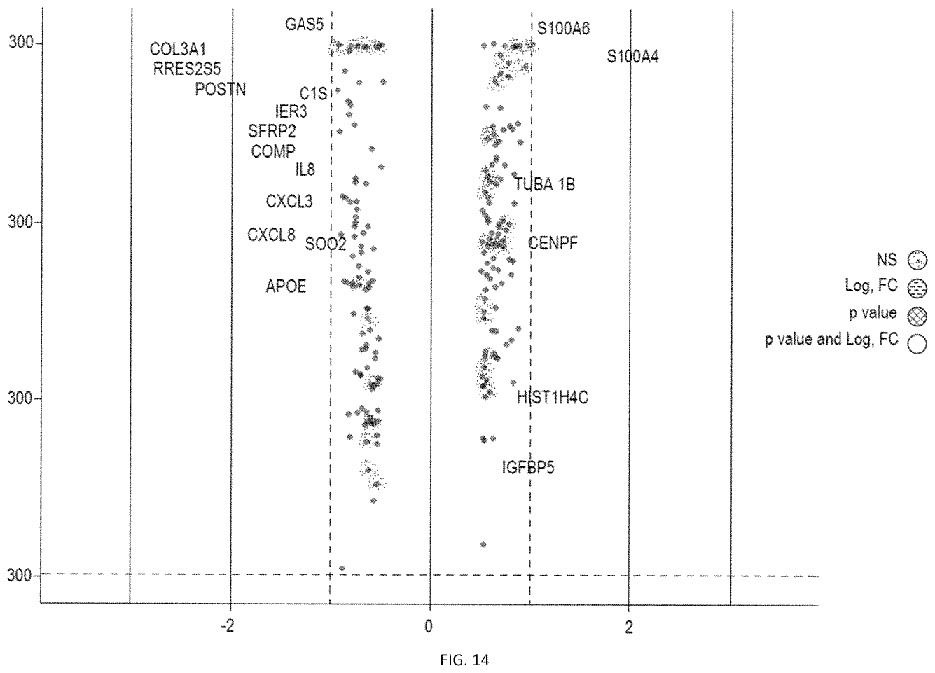

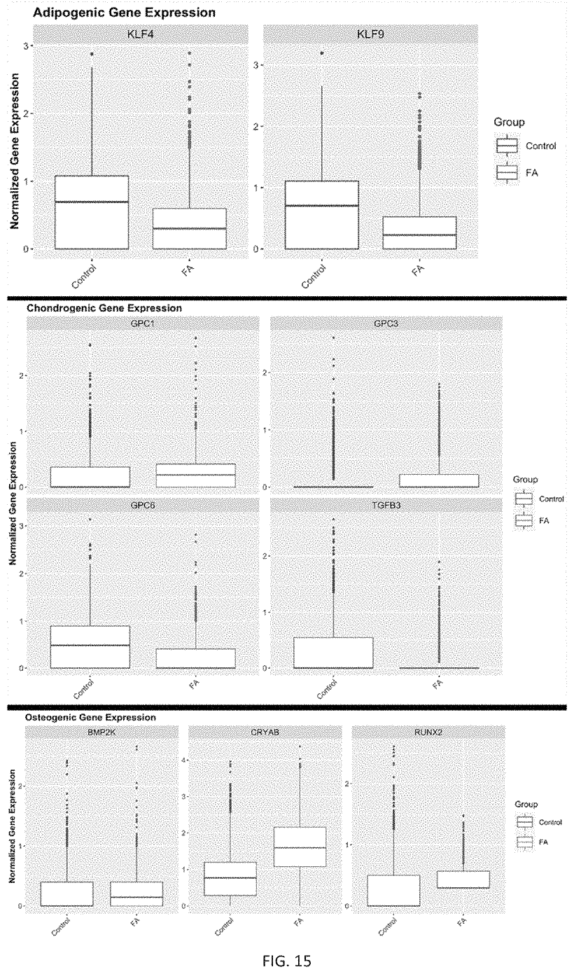

FIG. 14 is a volcano plot showing the differentially expressed genes between FA and control. FIG. 15 is a set of box plots showing the most differentially expressed genes expressed by stromal vascular fraction seeded on the FA and control surfaces, post 11 days of culturing. FIG. 14 shows that 261 genes were differentially expressed on FA surfaces compared to the control. Of those, mRNAs that are specific for osteogenic, adipogenic, and chondrogenic markers, as shown are given in FIG. 15. While adipogenic and chondrogenic markers are downregulated on FA surfaces, some osteogenic markers are upregulated. Specifically, within the osteogenic panel, there was a downregulation in collagen (COLA1 and COL2A2, which are also expressed by fibroblasts, not shown) gene expressions and upregulation in a stem cell bone differentiation gene (CRYAB)3, 4, and pre-osteoblast gene RUNX in stromal vascular fraction cells grown on FA compared to control. This data suggests a subset of cells present with stromal vascular fraction preferentially grow on FA, and there are early indications that more stromal vascular fraction cells may be transforming into an osteogenic lineage.

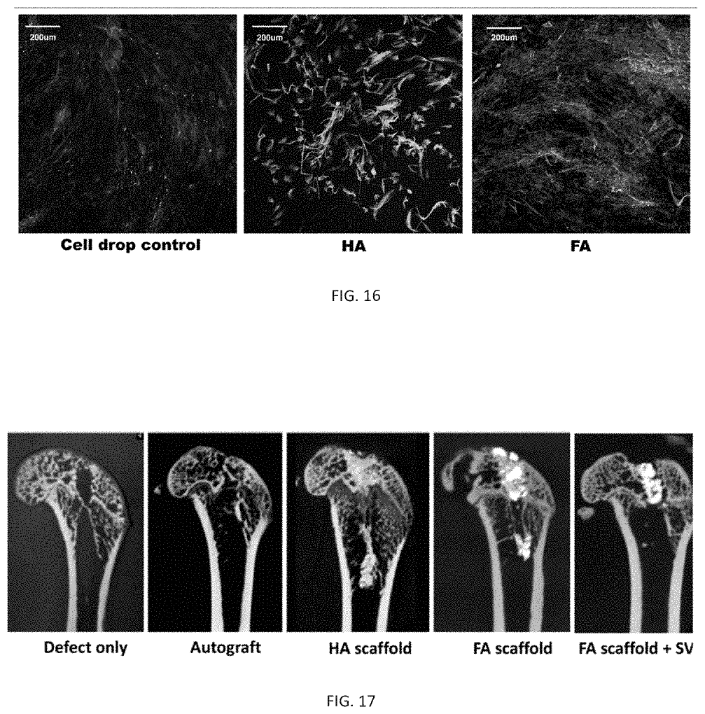

Based on the single-cell sequencing data, another cell-culture study was conducted to look at cell differentiation at a later time point, i.e., 11 days compared to 6 days post-seeding. As previously described, human adipose tissue was harvested and processed, and the stromal vascular fraction was characterized using FACS. Then equal numbers of stromal vascular fraction/area were seeded onto FA, HA, and cell culture plates, with the apatites sintered at 1150° C. The cells were cultured for 11 days and stained for osteoblast markers, osteopontin, and osteocalcin immunohistochemically. They were then imaged using confocal microscopy. FIG. 16 is representative images showing osteoblast marks, osteocalcin (OCN) and osteopontin (OPN) expressed after 11 days-post culturing. The greatest positive signals for both OPN and OCN were observed on cells that were plated onto the FA scaffolds compared to the HA and control surface, as shown in FIG. 16. In other words, when compared to the cell drop control, more OCN and OPN signals were present on apatite surfaces. Overall, there was greater expression of OCN and OPN on the FA surfaces. The earlier expression of osteoblast markers on FA compared to HA is in alignment with our observations when ADSC cells are grown on FA and HA. Through these in vitro studies, optimized the procedure for adipose tissue harvest and extraction of stromal vascular fraction have been obtained. It has also demonstrated that FA surfaces indeed induce differentiation of stem cells towards an osteogenic lineage without adding growth factors or hormones. This is an important finding, making the FA scaffold a unique bone scaffold.

### In Vivo Study Relating to Stromal Vascular Fraction

Thirty male Wistar rats were used for testing the efficacy of the scaffold to allow deposition of new bone. The rats were divided into four groups of six animals. Each animal received one of the following treatments (n=6): Group 1—defect left untreated (negative control), Group 2—defect filled with autograft bone (gold standard), Group 3—defect filled with HA scaffold, Group 4—defect filled with FA scaffold, and Group 5—defect filled with FA and 1·10⁶ of stromal vascular fraction cells. During a sterile surgery, a longitudinal incision was made on the lateral surface of the patellar tendon to expose the femoral condyle. A bone defect was then created in the center of the condyle and parallel to the long of the femur using a drill/needle and saline flush (approximately 2 mm wide and 4 mm long). The drilled cavity was flushed with saline. Animals were treated as assigned. The incision was closed with sutures. Every two weeks, all animals were subjected to in situ micro-CT scans to monitor the progress of bone regeneration. All animals remained in the study for 12 weeks post-surgery without any adverse events. At necropsy, the scaffolding and the surrounding tissues were harvested and subjected to histological analyses and micro-CT imaging. The volume data that was calculated from the micro-CT scans showed that new bone formed preferentially around and in-between the spaces occupied by the FA granules, while the defect-only remained unfilled.

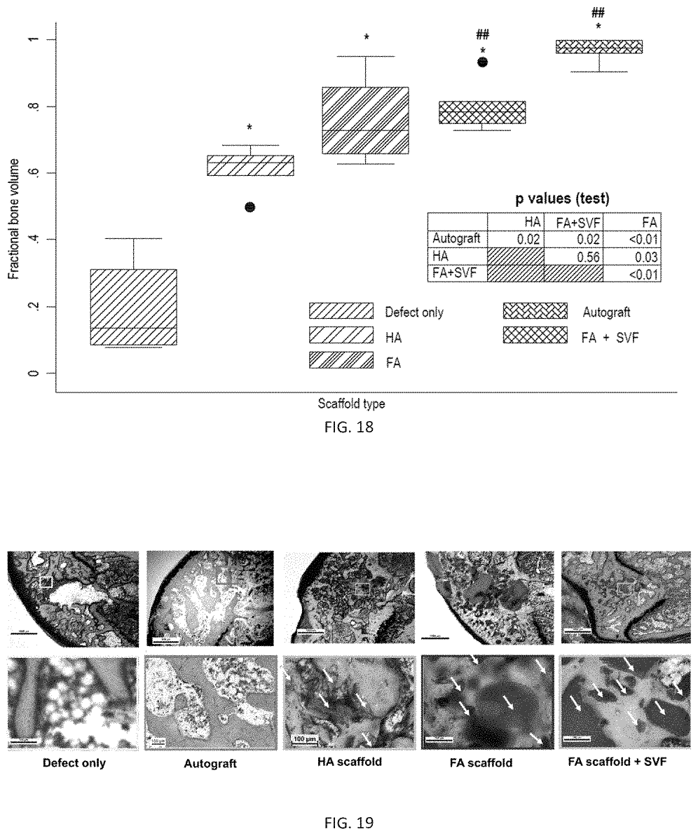

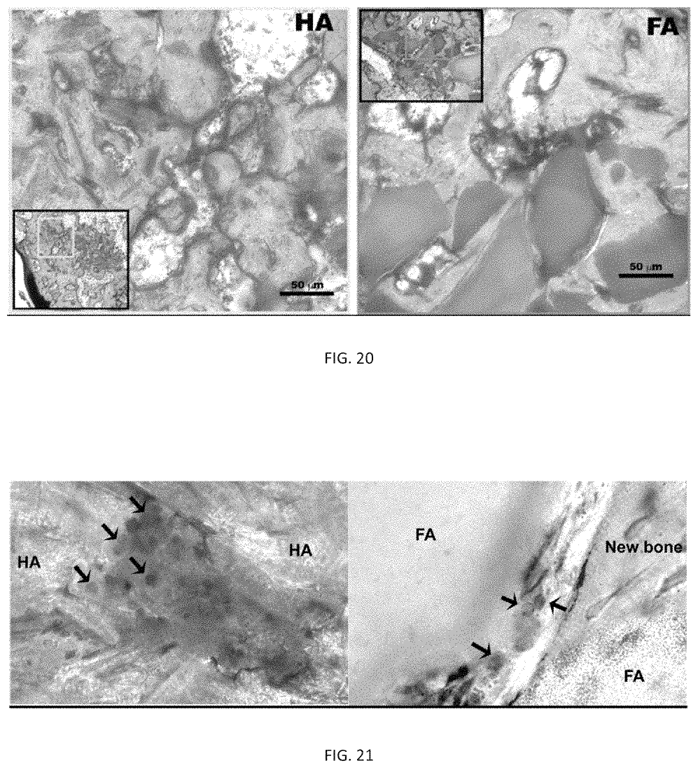

FIG. 17 is a representative set of micro-CT images of the femoral defect at necropsy (12 weeks, post-implantation). FIG. 17 demonstrates that, while the defect-only remained unfilled, the HA scaffolds group appeared to have no scaffolding material but filled bone and cellular infiltrates. Using an HA calibration standard, volumes of the new bone, scaffolding, and empty spaced within the defect regions were calculated, as shown in FIG. 18 (a box plot showing the percent of new bone within the defect). Although it is a semi-quantitative analysis, FIG. 18 overwhelmingly supports the ability of the FA scaffold to regenerate bone tissue. In the FA group, nearly 90% of the pores were filled with new bone, which is statistically different from all tested groups. Although total bone volume is a useful indicator, its relevance can only be interpreted in conjunction with histological findings, as shown in FIGS. 19-21. FIG. 19 is a representative set of photomicrographs of bone defects with and without scaffolding stained with Sanderson's Rapid Bone Stain™ and magnified images of the top panel are given in the bottom panel. FIG. 19 shows that the pores of the FA scaffold groups were completely filled with new-bone while the defect only showed bone formation on the perimeters of the defects. HA scaffold only group of FIG. 19 clearly shows the region of resorbed scaffolds with residual needle-like HA. FA scaffold (shown with arrows in FIG. 19) within and without stromal vascular fraction appear filled with new bone. Pink regions represent the mineralized bone tissue, and the blue areas represent the fiber-rich cartilage, cell nuclei, or bone marrow cells. The unstained white circles are fat cells. FIG. 20 is representative photomicrographs showing the multi-nucleated cells (possible osteoclasts, shown with arrows) adjacent to the FA surface (right) and a representative microphotograph of the resorbing (degraded) HA scaffolding (left), 12 weeks post-implantation. The right image of FIG. 20 potentially indicates the resorption mechanism of FA may involve osteoclastic activities. The left image of FIG. 20 shows direct connection to the intra-trabecular spaced and scaffolds where a dense community of multi-nucleated cells are seen in HA group. FIG. 21 is representative highly magnified photomicrographs showing the multi-nucleated cells osteoclast (indicated with arrows) adjacent to the FA surface (right) and within a cluster of HA surfaces (left). Importantly, these histological images of FIGS. 19-21 show that none of the tested scaffolds induced the formation of a fibrous capsule, indicating the absence of foreign body response (i.e., biocompatible). However, it should be emphasized that relatively fewer intra-trabecular spaces were found within the FA scaffold-only group. This perhaps exacerbated the difference in the micro-CT volume data of FIG. 18. In the FA and the stromal vascular fraction group, remodeling progressed and resulted in the identical microstructure to the trabecular bone with intra-trabecular spaces filled with fat cells. At the same time, HA scaffolds had clusters of immune cells and appeared to be resorbing (FIGS. 20 and 21). Another observation is that autograft bone appeared to maintain the trabecular microstructure of the surrounding bone tissue. It appears that autografts are simply remodeled and incorporated into the surrounding cancellous tissue. This observation partly explains why a lower bone volume was detected in micro-CT analysis, as shown in FIG. 18.

### Implantable Scaffolds Including the Metal Substitute

TABLE 1

| | | | Percent Molar Zn |
|---|---|---|---|
| Powder | Ca (M/kg) | Zn (M/kg) | Substitution |
| FA | 9.8 | 0 | 0 |
| 0.5% Zn-FA | 9.7 | 0.05 | 0.5 |
| 1.0% Zn-FA | 9.5 | 0.10 | 1.1 |
| 2.0% Zn-FA | 9.2 | 0.20 | 2.1 |

An FA powder and three zinc-substituted fluorapatites (0.5% Zn-FA, 1.0% Zn-FA, and 2.0% Zn-FA) powders were synthesized using a chemical co-precipitation technique. It is noted that the percentages used in this section refers to molar percent. Each of the powders were characterized using inductively coupled plasma mass spectrometry (ICP-MS) to determine the phosphate, zinc, and calcium contents of the powders, an x-ray diffraction technique was used to quantify the crystallography of the powders, and a fluoride probe to determine the fluoride content of the powders. Table 1 shows the molar weight of calcium and zinc in the synthesized powders and the calculated ICP-MS as percent molar substitution of zinc within the apatite crystal structure.

In a first set, the powders were compressed into 10 mm disks using a compressive load of about 31 MPa, about 62 MPa, and 94 MPa (i.e., a compressive force of 1,000 kg, 2,000 kg, and 3,000 kg, respectively). Initially, compression pressures were also altered to produce uniform surface micro-features. Compressed and sintered disks were imaged under a scanning electron microscope to document the surface microstructures. In the second set, the powders were formed into scaffolds using a foam-casting technique.

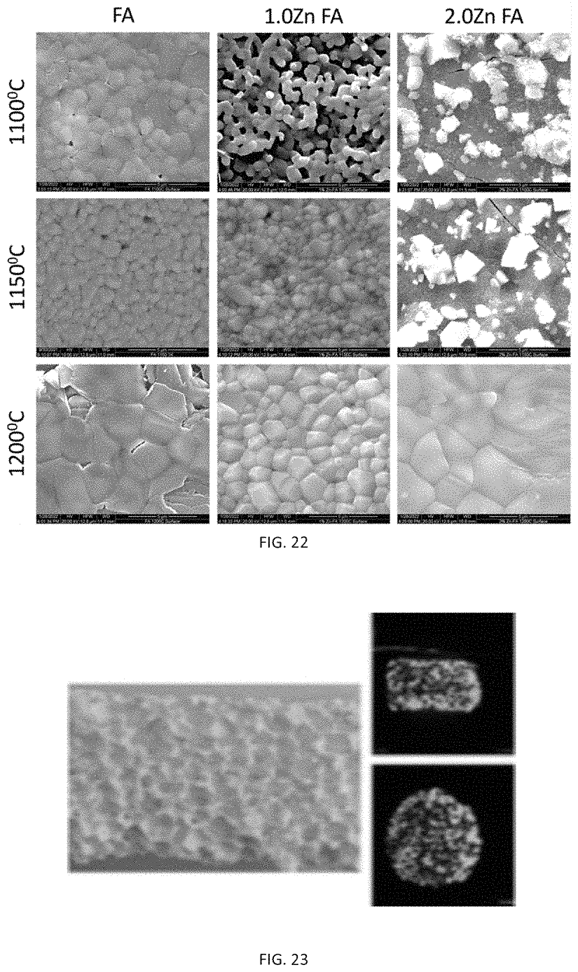

Each of the disks were sintered at 1100° C., 1150° C., and 1200° C. FIG. 22 is a representative set of SEM images showing FA and 1% and 2% Zn-doped foam-casted FA surfaces sintered at 1100° C., 1150° C., and 1200° C. FIG. 22 appears to demonstrate that 2% ZN-FA powders may require a higher temperature to form surface micro-features. FIG. 23 is a representative photograph of the foam-casted porous FA scaffolding (left) and two 2D slices from a micro-CT scan. FIG. 23 shows that the FA exhibited an open porous structure with interconnecting pores. The foam-casted FA exhibited about 60±12% porosity. Post-characterization, uniaxial compression strengths of the foam-casted scaffolds were calculated using a universal tensiometer. The compression strength of the foam-casted scaffolds sintered at 1100° C. ranged from 4.17 to 13.5 MPa, which is comparable to cancellous bone tissues. FIG. 24 is representative SEM images showing the surface morphology of several of the disks sintered at 1150° C. having different zinc content and different compressive loads.

Phosphate, zinc, and calcium content was determined with ICP-MS. ICP-MS showed a slightly lower Ca/P ratio at 1.42, compared to the expected value of stoichiometric FA, which is 1.67. The Ca/P ratio for 0.5, 1.0, and 2.0% Zn-FA was also lower than the expected value. The zinc substitution values for these powders were found to be at the expected substitution values. The lower Ca/P ratio may be attributed to the technique used for the synthesis,

### In Vitro Assays Involving Zinc Substituted Apatite

Cell viability studies were carried out using ADSCs maintained in growth media supplemented with 10% heat-inactivated fetal bovine serum, and incubated for 2 or 10 days on a control, FA scaffold sintered at 1150° C., and 2.0% Zn-FA scaffold sintered at 1150° C. All experiments were conducted with cells between passages 3-5. ADSCs were plated onto the different surfaces and then evaluated for cell viability with alamarBlue®. All data are reported as mean±SD and relative to cells grown on a cell culture plate (n=4/disks/group/assay). Group differences in ADSC viability and were evaluated by ANOVA, followed by a Tukey's post hoc test (JMP, SAS Institute). Significance was set at p<0.05. After 2 and 10 days of incubation, no significant difference in cell viability (p>0.05) was seen between the FA disks and the 2.0 molar % Zn-FA disks. In other words, the data showed that sintered, 2.0 molar % Zn-FA disks had no difference in cell toxicity when compared to an FA disks. ADSC adhesion assays showed that there were no differences between FA and 2% Zn-FA in cell adhesion numbers after 2 and 10 days of incubation.

Initial trials showed that the compressive loads used to form the disks affected the surface morphology and the microstructures which, in turn, affected the number of bacteria colonies that were adhered to the disks. For example, it was found that increasing the compressive loading used to form the disks significant decreased (e.g., by a 1 to 2 log-fold decrease) the bacterial adhesion on the disks. The disks pressed with higher compressive loads had smaller microstructural features and the least numbers of bacterial adhesion compared to disks pressed with lower compressive loads. This observation was made independent of sintering temperature. Furthermore, the biofilm data indicated the least amount of bacteria adhered to the disks sintered at 1150° C. when compared to other temperatures studied after a 48 hour period of incubation in the biofilm reactor. This sintering temperature was then used for fabricating porous scaffolding for in vivo testing. It was also found that adding zinc to the FA crystal structure decreased bacterial adhesions with the 2.0% Zn-FA substitution resulting in better antimicrobial surface. In particular, the 2.0 molar % Zn-FA disks have a 1 to 2-log fold reduction is bacterial adhesion when compared to the FA disks.

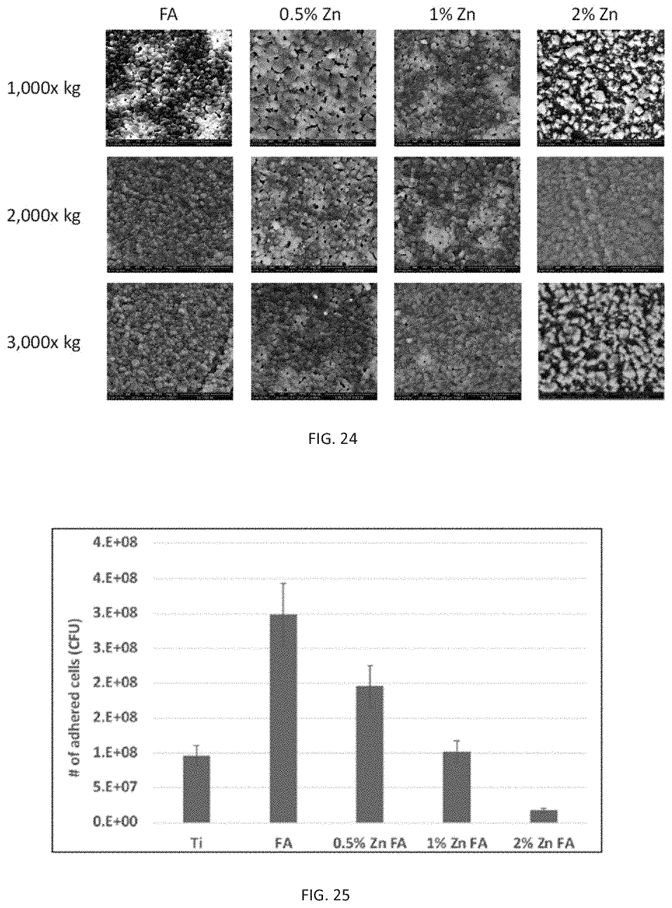

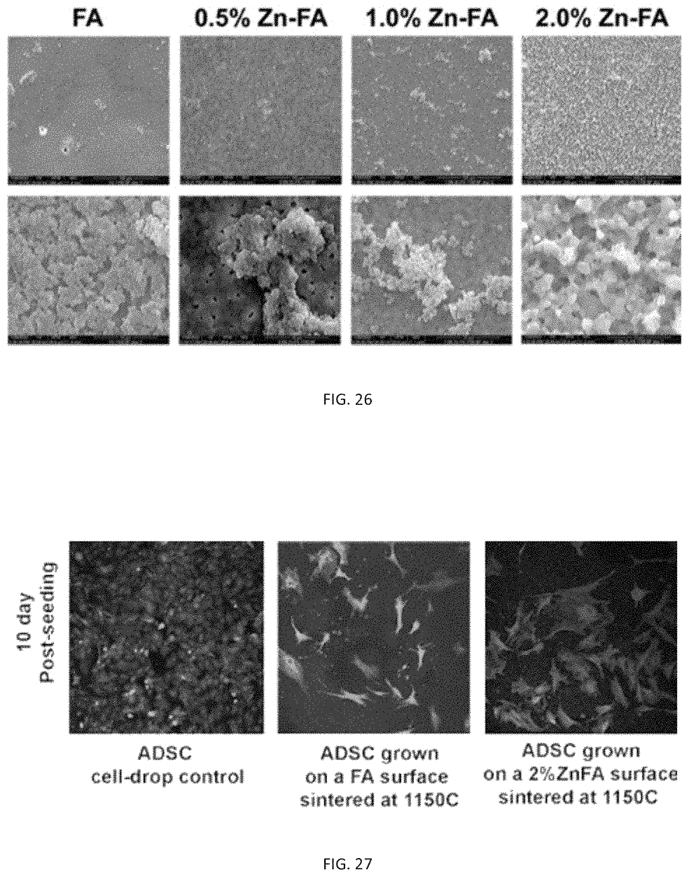

Another study included evaluating the abilities of zinc substituted apatite to limit bacterial adhesion in a highly contaminated environment following ASTM standard protocol (ASTM E3161-18) with Staphylococcus aureus (ATCC 6538; S. aureus). For this study, 10 mm disks were fabricated by compressing a known weight (0.3 g) of apatites and then sintered at 1100, 1150, or 1200° C. Some of the disks were then processed to quantify bacteria on the surface and others for SEM imaging. FIG. 25 is a bar chart showing the number of adherent bacteria on zinc substituted disks sintered at 1150° C. after 48 hours in a biofilm reactor. FIG. 25 shows that the 2% Zn-FA had a log fold reduction in adhered bacteria (5.4×106 CFU) compared to the titanium disks (2.99×10⁷ CFU), and 1-log fold reduction compared to the FA (1.92×10⁷ CFU). FIG. 26 is representative SEM images showing the adherent biofilm after 48 hours in a biofilm reactor. The images in the top row were taken at 500× magnification and the images on the bottom row were taken at 5000 times magnification. The images of FIG. 26 shows that fewer bacteria remained on the 2.0% Zn-FA surfaces than any of the other surfaces. The osteogenic properties of the sintered 2.0% Zn-FA surfaces using adipose-derived stem cells (ADSC) were then determined. At ten days post-plating, there were no significant differences in cell viability between ADSC seeded on FA scaffold (1.05±0.01; data normalized to cell plate control) or 2% Zn-FA (1.07±0.01; p=0.52), which indicates the introduction of zinc did not adversely affect the cell growth (biocompatibility). FIG. 27 is a representative set of confocal images showing the nucleus and osteocalcin 10-days post-seeding. The confocal images of FIG. 27 clearly show that the introduction of zinc did not appear to prevent the surface-induced cellular differentiation properties of the apatites compared to FA. In fact, ADSCs growing on FA and 2% Zn-FA sintered at 1150° C. appear to have a greater expression of late OCN.

### In Vivo Studies Involving Zinc Substituted Apatites

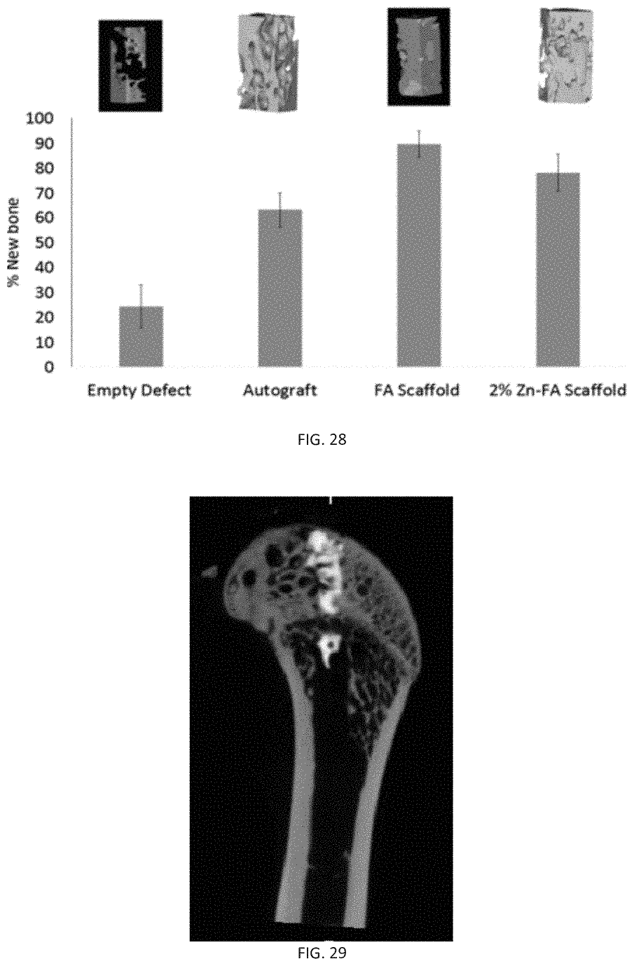

A foam-casting technique was used to fabricate scaffolds with aqueous slurries of Zn-FA powder, which was formulated using distilled water, polyvinyl alcohol, and polyethylene glycol as binders. The foam-casting templates were dried and sintered at 1150° C. Resultant porous implantable scaffolds were cleaned sterilized, and implanted in a rat femoral intra-condyle bond defect model for twelve weeks (n=6/group). The groups were 1) empty defect left untreated (negative control), 2) defect filled with autograft (current gold standard, positive control), 3) defect filled with FA scaffold, and 4) defect filled with 2% Zn-FA scaffold. Every two weeks, all animals were subjected to in situ micro-CT scans to monitor the progress of boned regeneration. FIG. 28 is a bar graph showing the percent of new bone within the scaffolding after 12 weeks in situ in rats. FIG. 28 shows the enhanced ability of the FA and 2% Zn-FA scaffold to regenerate bone tissue when compared to autograft (statistically significant p<0.05). Within the 2% Zn-FA scaffolds, nearly 72% of the pores were filled with new bone tissues. Histology supported the micro-CT data. Thus, it was concluded that incorporating 2% zinc apatites to the FA matrices did not impede FA scaffolding's ability to regenerate bone tissue.

FIG. 29 is a representative micro-CT image of 2.0 molar % Zn-FA scaffold encapsulated by bone matrix post-10 weeks in situ. The 2.0 molar % Zn-FA scaffold was fully encapsulated within the bone matrix after 10 weeks in situ. In vivo data further confirmed that there were no biocompatibility issues associated with including a lower percentage Zn-substitution in FA.

The data obtained from the zinc-substituted FA disks shows that by substituting zinc into the apatite crystallite structure, the antimicrobial properties of FA can be improved without forgoing its osteogenic abilities. It is believed that such substitutions can impart protection during the lifetime of the bone scaffolds themselves.

While various aspects and embodiments have been disclosed herein, other aspects and embodiments are contemplated. The various aspects and embodiments disclosed herein are for purposes of illustration and are not intended to be limiting.

1. An implantable scaffold, comprising:
a fluoridated apatite structure sized and shaped for implantation in an animal, the fluoridated apatite structure defining a plurality of pores;
at least one integration aid including at least one of:
a stromal vascular fraction adhered to the fluoridated apatite structure; or
at least one metal substitute substituted into the fluoridated apatite structure, the at least one metal substitute including one or more of zinc, silver, or iron.

2. The implantable scaffold of claim 1, wherein the fluoridated apatite structure includes at least one of fluoridated apatite or fluorohydroxyapatite.

3. The implantable scaffold of claim 1, wherein the at least one integration aid includes the stromal vascular fraction.

4. The implantable scaffold of claim 3, wherein the stromal vascular fraction includes adipose tissue-derived stromal vascular fraction.

5. The implantable scaffold of claim 3, wherein the stromal vascular fraction includes perivascular cells, leukocytes, endothelial cells, fibroblasts, and progenitor stem cells.

6. The implantable scaffold of claim 3, wherein the stromal vascular fraction includes adipose derived stem cells.

7. The implantable scaffold of claim 1, wherein the at least one integration aid includes the at least one metal substitute substituted into the fluoridated apatite structure.

8. The implantable scaffold of claim 7, wherein the at least one metal substitute includes zinc.

9. The implantable scaffold of claim 7, wherein about 0.25 molar % to about 4 molar % of calcium in the fluoridated apatite structure is replaced with the at least one metal substitute.

10. The implantable scaffold of claim 7, wherein about 0.5 molar % to about 5 molar % of calcium in the fluoridated apatite structure is replaced with the at least one metal substitute.

11. (canceled)

12. (canceled)

13. The implantable scaffold of claim 7, wherein the at least one integration aid further includes the stromal vascular fraction.

14. The implantable scaffold of claim 1, further comprising one or more dopant disposed in or on the fluoridated apatite structure, the one or more dopants including one or more stem cells.

15. A method of making an implantable scaffold, the method comprising:
providing fluoridated apatite particles;
sintering the fluoridated apatite particles at a sintering temperature of at least 950° C. to form a fluoridated apatite structure; and
introducing at least one integration aid into the fluoridated apatite structure, the at least one integration aid includes at least one of:
a stromal vascular fraction adhered to at least a portion of the fluoridated apatite structure; or
at least one metal substitute substituted into the fluoridated apatite structure, the at least one metal substitute including one or more of zinc, iron, or silver.

16. (canceled)

17. The method of claim 15, wherein the at least one integration aid includes the stromal vascular fraction adhered to at least a portion of the fluoridated apatite structure.

18. (canceled)

19. (canceled)

20. (canceled)

21. (canceled)

22. The method of claim 17, wherein introducing at least one integration aid into the fluoridated apatite structure includes, during a procedure to implant the implantable scaffold in the subject:
removing adipose tissue from the subject;
isolating the stromal vascular fraction from the adipose tissue; and
adhering the stromal vascular fraction to the fluoridated apatite structure.

23. The method of claim 15, wherein the at least one integration aid includes the at least one metal substitute substituted into the fluoridated apatite structure.

24. (canceled)

25. (canceled)

26. (canceled)

27. (canceled)

28. (canceled)

29. The method of claim 15, wherein providing fluoridated appetite particles includes forming the fluoridated apatite particles by substituting the at least one metal substitute into the fluoridated apatite particles.

30. (canceled)

31. A method of using an implantable scaffold, the method comprising:
providing an implantable scaffold, the implantable scaffold including:
a fluoridated apatite structure sized and shaped for implantation in an animal, the fluoridated apatite structure defining a plurality of pores;
at least one integration aid including at least one of:
a stromal vascular fraction adhered to at least a portion of the fluoridated apatite structure; or
at least one metal substitute substituted into the fluoridated apatite structure, the at least one metal substitute including one or more of zinc, iron, or silver; and
implanting the implantable scaffold in a subject.

32. (canceled)

33. The method of claim 31, wherein the at least one integration aid includes the stromal vascular fraction adhered to at least a portion of the fluoridated apatite structure.

34. (canceled)

35. (canceled)

36. (canceled)

37. (canceled)

38. The method of claim 31, further comprising, during a procedure to implant the implantable scaffold in the subject and before implanting the implantable scaffold in the subject:
removing adipose tissue from the subject;
isolating the stromal vascular fraction from the adipose tissue; and
adhering the stromal vascular fraction to the fluoridated apatite structure.

39. The method of claim 31, wherein the at least one integration aid includes the at least one metal substitute substituted into the fluoridated apatite structure.

40. (canceled)

41. (canceled)

42. (canceled)

43. (canceled)

44. (canceled)
